(19) United States
(12) Patent Application Publication
(10) Pub. No.: US 2023/0357722 A1
(43) Pub. Date: Nov. 9, 2023

(54) METHODS FOR IDENTIFYING MODULATORS OF NATURAL KILLER CELL INTERACTIONS

(71) Applicant: The Johns Hopkins University, Baltimore, MD (US)

(72) Inventors: Isaac C. Chan, Baltimore, MD (US); Andrew J. Ewald, Baltimore, MD (US)

(21) Appl. No.: 18/029,750

(22) PCT Filed: Oct. 1, 2021
(86) PCT No.: PCT/US2021/053200

§ 371 Date: Mar. 31, 2023

Related U.S. Application Data
(60) Provisional application No. 63/087,063, filed on Oct. 2, 2020.

(51) Int. Cl. C12N 5/0783; G01N 33/50

(52) U.S. Cl. CPC C12N 5/0646; G01N 33/5011; C12N 2502/30; C12N 2533/90; C12N 2513/00; G01N 2800/52; C12N 2503/02

(57) ABSTRACT

Disclosed herein, are methods for identifying a drug candidate for treating cancer metastasis. The method comprising culturing an embedded organoid/natural killer (NK) cell co-culture with a drug candidate; and measuring one or more metastatic properties of the embedded organoid or tumor-killing potential or tumor-promoting potential of the NK cells in the co-culture.

### CROSS REFERENCE TO RELATED APPLICATIONS

This application claims the benefit of U.S. Provisional Application No. 63/087,063, filed on Oct. 2, 2020. The content of this earlier filed application is hereby incorporated by reference herein in its entirety.

### GOVERNMENT SUPPORT

This invention was made with government support under grant CA217846 awarded by the National Institutes of Health. The government has certain rights in the invention.

### INCORPORATION OF THE SEQUENCE LISTING

The present application contains a sequence listing that is submitted via EFS-Web concurrent with the filing of this application, containing the file name “36406_0022P1_SL.txt” which is 4,096 bytes in size, created on Oct. 1, 2021, and is herein incorporated by reference in its entirety.

### BACKGROUND

Metastatic disease is the major driver of breast cancer mortality (Siegel R L, et al. C A Cancer J Clin. 2017; 67(1):7-30). Adjuvant chemotherapy is used after locoregional control to prevent metastatic recurrence but it is not sufficiently effective because how metastases form is not fully understood. While the loss of immunosurveillance is important to breast cancer metastasis, immune checkpoint blockade has not been as effective in treating metastatic breast cancer as in melanoma or lung cancer (Adams S, et al. JAMA Oncol. 2019). This clinical observation suggests that the tumor microenvironment in metastatic breast cancer is complex and that inhibiting PD-1/PD-L1 signaling is not sufficient to restore a robust anti-tumor immune response. Thus, alternative treatment strategies are needed.

### SUMMARY

Disclosed herein are compositions comprising an organoid/natural killer (NK) cell co-culture, wherein the organoid/NK cell co-culture comprises one or more tumor organoids with one or more NK cells embedded in an extracellular matrix.

Disclosed herein are methods of identifying a drug candidate for treating cancer metastasis, the methods comprising: a) culturing an embedded organoid/natural killer (NK) cell co-culture with a drug candidate; and b) measuring one or more metastatic properties of the embedded organoid or tumor-killing potential or tumor-promoting potential of the NK cells in the co-culture, wherein a change in the one or more metastatic properties or an increase in the tumor killing tumor potential of the NK cells or a decrease in the tumor promoting potential in the NK cells identifies the drug candidate is capable of treating cancer metastasis.

Disclosed herein are methods of identifying a drug candidate that inhibits natural killer cell activity, the methods comprising: a) culturing an embedded organoid/NK cell co-culture with a drug candidate; and b) measuring one or more metastatic properties of the embedded organoid or tumor-killing potential or tumor-promoting potential of the NK cells in the co-culture, wherein a change in the one or more metastatic properties or an increase in the tumor killing tumor potential of the NK cells or a decrease in the tumor promoting potential in the NK cells identifies a drug candidate capable of inhibiting natural killer cell activity.

Disclosed herein are methods of identifying a cancer patient's responsiveness to an anticancer drug candidate, the methods comprising: a) culturing an embedded organoid/NK cell co-culture with a drug candidate; and b) measuring one or more metastatic properties of the embedded organoid or tumor-killing potential or tumor-promoting potential of the NK cells in the co-culture, wherein a change in the one or more metastatic properties or an increase in the tumor killing tumor potential of the NK cells or a decrease in the tumor promoting potential in the NK cells identifies an anticancer drug that the subject is responsive to.

Disclosed herein are methods identifying an antibody that binds to a specific tumor antigen, the methods comprising: a) culturing an embedded organoid/natural killer (NK) cell co-culture with an antibody; and b) measuring one or more metastatic properties of the embedded organoid or tumor-killing potential or tumor-promoting potential of the NK cells in the co-culture, wherein a change in the one or more metastatic properties or an increase in the tumor killing tumor potential of the NK cells or a decrease in the tumor promoting potential in the NK cells means that the antibody binds to the specific tumor antigen.

Disclosed herein are methods identifying a drug candidate for treating cancer metastasis, the methods comprising: a) culturing an embedded organoid/natural killer (NK) cell co-culture with a drug candidate; and b) measuring one or more metastatic properties of the embedded organoid or tumor-killing potential or tumor-promoting potential of the NK cells in the co-culture, wherein a change in the one or more metastatic properties or an increase in the tumor killing tumor potential of the NK cells or a decrease in the tumor promoting potential in the NK cells means that the antibody binds to the specific tumor antigen.

Disclosed herein are methods of identifying a drug candidate for treating cancer metastasis, the methods comprising: a) culturing an embedded organoid/natural killer (NK) cell co-culture with a drug candidate; and b) measuring one or more metastatic properties of the embedded organoid or tumor-killing potential or tumor-promoting potential of the NK cells in the co-culture, wherein the one or more tumor organoids comprise one or more natural killer cells with Klrg1, TIGIT, or Lag3 present on its surface; wherein a change in the one or more metastatic properties or an increase in the tumor killing tumor potential of the NK cells or a decrease in the tumor promoting potential in the NK cells means that the drug candidate inhibits binds to Klrg1, TIGIT, or Lag3 present on the surface of the one or more natural killer cells and wherein the wherein the change in the one or more metastatic properties or an increase in the tumor killing tumor potential of the NK cells or a decrease in the tumor promoting potential in the NK cells indicates that the subject is responsive to treatment with the drug.

### BRIEF DESCRIPTION OF THE DRAWINGS

FIGS. 1A-H show NK cells limit early stages of metastasis in ex vivo models of breast cancer. FIG. 1A shows a dot plot of GFP+K14+ and K14− tumor cells stained for MHC class I expression. FIG. 1B shows a schema of hNK cell-tumor organoid co-culture. Tumor organoids were isolated from dissected MMTV-PyMT mammary tumors and hNK cells were isolated from the spleens of FVB/n mice. Tumor organoids were cultured alone or in co-culture with hNK cells in collagen I gels. FIG. 1C shows representative DIC images of MMTV-PyMT tumor organoids alone (top) or in co-culture with hNK cells (bottom) at 0 hours and 24 hours. Scale bar, 50 μm.

FIGS. 1D-E show boxplots of (FIG. 1D) inverse circularity of MMTV-PyMT tumor organoids alone or in co-culture with hNK cells and (FIG. 1E) area fold change of MMTV-PyMT tumor organoids alone or in co-culture with hNK cells. Error bars represent 5^{th} to 95^{th} percentile. ****p-value<0.0001 by the Mann-Whitney test. FIG. 1F shows a schema of hNK cell-tumor clusters co-culture. Tumor clusters were isolated from dissected MMTV-PyMT mammary tumors and hNK cells are isolated from the spleens of WT mice. Tumor clusters are cultured alone or in co-culture with hNK cells in Matrigel. FIG. 1G shows representative DIC images of MMTV-PyMT tumor colonies alone (top) or in co-culture with hNK cells (bottom) at 24 hours. Scale bar, 50 μm. FIG. 1H shows the quantification of normalized colony formation count from MMTV-PyMT tumor clusters cultured alone or in co-culture with hNK cells. Colony count was normalized to control. Mean is represented and ***p-value<0.001 by the Mann-Whitney test.

FIGS. 2A-J show that healthy NK cells induce apoptosis in K14+ invasive breast cancer cells. FIG. 2A shows representative confocal images of the invading strands of tumor organoids (mTomato+), and caspase activity (green), when (top) cultured alone or (bottom) co-cultured with hNK cells. Scale bar, 10 μm. FIGS. 2B-C show a boxplot of (FIG. 2B) the percentage of organoids with caspase activity in invading strands per biological replicate, and (FIG. 2C) the total number of invading strands with caspase activity, when cultured alone or co-cultured with hNK cells. FIG. 2D shows representative confocal images of tumor clusters organoids (mTomato+), and caspase activity (green), when (top) cultured alone or (bottom) co-cultured with hNK cells. Scale bar, 10 μm. FIG. 2E shows a boxplot of the percentage of tumor clusters per biological replicate with caspase activity in tumor clusters when cultured alone or co-cultured with hNK cells. FIG. 2F shows a schema for assessing interferon-gamma (IFNγ) activity within hNK cells in response to co-culture with K14+ or K14− tumor cells. hNK cells are taken from ROSA^{mT/mG} mice and co-cultured with K14+ or K14− cells from K14-actin-GFP; MMTV-PyMT mice. In this experiment, hNK cells are fluorescently labeled with mTomato while K14+ cells are labeled with GFP. FIG. 2G shows a boxplot of IFNγ expression among hNK cells after co-culture with K14+ and K14− cells and normalized to K14− cells. Error bars represent 5^{th} to 95^{th} percentile. ***p-value<0.001 by the Mann-Whitney test. FIG. 2H shows a schema for assessment of the innate immune response to an initial metastatic seed. Tumor clusters from the mammary tumors of K14-actin-GFP; MMTV-PyMT; ROSA^{mT/mG} mice were injected into the tail veins of immunocompetent mice and the lung microenvironment is assessed after 6 hours. FIG. 2I shows a boxplot of the number of NK cells, macrophages, and neutrophils around a metastatic seed. Error bars represent 5^{th} to 95^{th} percentile. ***p-value<0.001, ****p-value<0.0001 by the Kruskal-Wallis test. FIG. 2J shows representative slide scanned images of lung tissue field of view containing a K14+(green), metastatic seed (magenta), surrounded by NK cells (NK1.1, white). Scale bar, 20 μm.

FIGS. 3A-N show that tumor-exposed NK cells promote colony formation. FIG. 3A show a schema for teNK cell-tumor organoid co-culture. FIG. 3B shows a boxplot of tumor organoid invasion strands of tumor organoids cultured alone or in co-culture with teNK cells. Error bars represent 5^{th} to 95^{th} percentile. ns is not significant by the Mann-Whitney test. FIG. 3C shows a schema for teNK cell-tumor cluster co-culture. FIG. 3D shows normalized colony count of tumor clusters cultured alone or in co-culture with tumor-exposed NK (teNK) cells. Mean with SEM is represented. **p-value<0.01 by the Mann-Whitney test. FIG. 3E shows normalized colony count of tumor clusters cultured alone or in co-culture with tumor-infiltration NK (tiNK) cells. Mean with SEM is represented. *p-value<0.05 by the Mann-Whitney test. FIG. 3F shows a schema for generating culture-educated NK (ceNK) cells. FIG. 3G shows normalized colony count of MMTV-PyMT tumor clusters cultured alone or in co-culture with ceNK cells. Mean with SEM is represented. **p-value<0.01 by the Mann-Whitney test. FIG. 3H shows normalized colony count of MCF-7 cell clusters cultured alone or in co-culture with culture-educated human NK (ceHuNK) cells. Mean with SEM is represented. **p-value<0.01 by the Mann-Whitney test. FIG. 3I shows a schema of the adoptive transfer of NK cells following a tail vein injection of cancer cells. FIG. 3J shows representative whole lung images. Macrometastases were identified based on their mTomato expression. Scale bar, 4 mm. FIG. 3K shows a boxplot of the number of lung macrometastases. Error bars represent 5^{th} to 95^{th} percentile. *p-value<0.05, ****p-value<0.0001 by the Kruskal-Wallis test. FIG. 3L shows a heat map displaying z-scores for the variance-stabilized transform of gene expression for differentially expressed genes with absolute value of log 2(fold change) above 1 between healthy NK cells and tumor-exposed NK cells. Hierarchical clustering was used to order the genes. FIG. 3M shows a waterfall plot of genes associated with an active and resting NK cell phenotype, expressed by teNK cells and hNK cells. FIG. 3N shows the gene ontology enrichment analysis in ‘Biological Process’ category for differentially expressed genes up-regulated and down-regulated by tumor-exposed NK cells. Four categories with the lowest p-value related to the immune system, metabolic processes, apoptosis and proliferation are displayed.

FIGS. 4A-E show that the tumor-exposed NK cell phenotype can be reversed. FIG. 4A shows a heat map of z-scores of gene expression by healthy NK cells and tumor-exposed NK cells of genes related to NK cell inhibitory signaling. Hierarchical clustering was used to order the genes. FIG. 4B shows the relationship map of receptor-ligand pairs between teNK cells and K14+ or K14− cells as identified by the iTalk algorithm. FIGS. 4C-E show the normalized colony count from antibody treated control assays and tumor-exposed NK cell—MMTV-PyMT tumor cluster co-culture assays. Mean with SEM is represented. ns is not significant, *p-value<0.05, **p-value<0.01, ***p-value<0.001 by the Kruskal-Wallis test.

FIGS. 5A-E show that pretreatment of teNK cells with FDA approved DNMT inhibitors neutralizes the teNK cell phenotype. FIG. 5A shows the heat map of z-scores of gene expression by healthy NK cells and tumor-exposed NK (teNK) cells of genes related to DNA methyltransferases. Hierarchical clustering was used to order the genes. FIG. 5B shows a schema of pretreatment of teNK cells before co-culture with tumor clusters. FIG. 5C shows the normalized colony count from DMSO control or DNMT inhibitor pretreated teNK cell—MMTV-PyMT tumor cluster co-culture assays vs monoculture controls. Mean with SEM is represented. *p-value<0.05, **p-value<0.01 by the Mann-Whitney test. FIGS. 5D-E shows the normalized colony count from DMSO or DNMT inhibitor pretreated teNK cells and antibody treated monoculture control assays and tumor-exposed NK cell—tumor cluster co-culture assays. Mean with SEM is represented. *p-value<0.05, **p-value<0.01 by the Mann-Whitney test.

FIGS. 6A-D shows that healthy NK cells limit invasion, growth, and colony formation in the C3(1)-Tag mouse model of breast cancer. FIG. 6A shows representative DIC images of tumor organoids alone (top) or in co-culture with hNK cells (bottom) at 0 hours and 24 hours. Scale bar, 50 μm. FIGS. 6B-C shows a boxplot of (FIG. 6B) inverse circularity of tumor organoids alone or in co-culture with hNK cells and (FIG. 6C) area fold change of tumor organoids alone or in co-culture with hNK cells. Error bars represent 5^{th} to 95^{th} percentile. **p-value<0.01, ***p-value<0.001 by the Mann-Whitney test. FIG. 6D show the quantification of normalized colony counts from tumor clusters cultured alone or in co-culture with hNK cells. **p-value<0.01 by the Mann-Whitney test.

FIGS. 7A-E show that healthy NK cells induce caspase activity in K14+ invasive cells and healthy NK cell cytotoxicity can be increased by using a CD44 antibody specific to K14+ cells. FIG. 7A shows representative confocal images of tumor organoids and (FIG. 7B) tumor clusters stained for caspase activity (green) and K14 (white) among tumor organoids cultured alone (top) or in co-culture with hNK cells (bottom). Scale bar, 10 FIG. 7C shows representative confocal images of staining tumor cell clusters for CD44 and K14. Scale bar, 10 μm. FIG. 7D shows a schema for the antibody dependent cell mediated cytotoxicity assay. Tumor clusters were isolated from MMTV-PyMT mammary tumors and incubated with a CD44 antibody before being co-cultured with hNK cells at a reduced ratio of 10 NK cells to 1 tumor cell. FIG. 7E shows a boxplot of the normalized colony count. Error bars represent 5^{th} to 95^{th} percentile. ns is not significant, *p-value<0.05, ***p-value<0.001 by the Mann Whitney test.

FIGS. 8A-D show the quantification of macrophage and neutrophil response to early metastatic seeds in the lungs. FIG. 8A shows a schema for assessment of the innate immune response to an initial metastatic seed. Tumor clusters from the mammary tumors of K14-actin-GFP; MMTV-PyMT; ROSA^{mT/mG} mice are injected into the tail veins of immunocompetent mice and the lung microenvironment is assessed for macrophages and neutrophils after 6 hours. FIG. 8B shows a representative slide scanned images of early metastatic seeds staining for F4/80 (macrophages, white) and neutrophil-elastase (neutrophils, white) around K14+(green) metastatic seeds (magenta). Scale bar, 20 μm. FIG. 8C shows a schema for the control experiment where PBS is injected into immunocompetent host mice and the lung microenvironment is assessed for macrophages and neutrophils after 6 hours. FIG. 8D shows a representative slide scanned images of staining for NK1.1 (NK cells, white), F4/80 (macrophages, white) and neutrophil-elastase (neutrophils, white) around tumor clusters. Scale bar, 20 μm.

FIG. 9A-B show that breast cancer organoids are able to overcome hNK cell cytotoxicity over time in 3D culture. FIGS. 9A-B show representative tumor organoids isolated from MMTV-PyMT (FIG. 9A) and C3(1)-Tag mice (FIG. 9B) placed in 3D collagen I alone (top) or in co-culture with hNK cells from FBV/n mice (bottom). Although hNK cells are initially able to limit tumor organoid invasion in both models at 24 hours, by 36-48 hours, tumor organoids are able to invade despite hNK cell activity. Scale bar, 50 μm.

FIG. 10A-F show that RNA-seq analysis of healthy NK cells and tumor-exposed NK cells reveals differences in identity and biological processes. Receptor-ligand analysis of healthy NK cells and K14+ or K14− cells reveals interactions between NK cells and cancer cells. Treatment with DNMT inhibitors alter gene expression of inhibitory receptors. FIG. 10A shows a schema for RNA-seq analysis of hNK cells and teNK cells. FIG. 10B shows that Gene Ontology enrichment analysis in ‘Biological Process’ category for genes differentially expressed between hNK and teNK cells. 30 categories with the lowest p-value associated with up- or down-regulated tumor-exposed NK cells are displayed. FIGS. 10C-D show a network representation of total receptor-ligand pairs between hNK cells (FIG. 10C) or teNK cells (FIG. 10D) and K14+ or K14− cells as identified by the databases included in the iTalk algorithm. FIG. 10E shows a relationship map of receptor-ligand pairs of hNK cells and K14+ or K14− cells as identified by the databases included in the iTalk algorithm. FIG. 10F shows the treatment of teNK cells with azacitidine or decitabine alters gene expression of TIGIT and KLRG1 by qPCR.

FIG. 11 shows the results of applying a drug library to the co-culture system to enhance NK cell killing of cancer cells. Each dot is a compound. The x-axis represents each individual compound. The y-axis represents a calculated Z-score based on the luminescence of the cancer cells compared to the treated controls for each compound. The two dots above the dotted line showed highly significant enhancement of NK-mediated cell killing by two FDA approved oncology drugs identified in an unbiased and quantitative fashion.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been identified with a need for a treatment, such as, for example, prior to making the tumor organoids.

As used herein, the term “comprising” can include the aspects “consisting of” and “consisting essentially of.”

As used herein, the term “assay” is intended to be equivalent to “method”.

The term “organoid” refers to an in vitro collection of cells which resemble their in vivo counterparts and form 3D structures.

The term “resembles” means that the organoid has genetic and phenotypic characteristics that allow it to be recognized by the skilled person as being from or associated with a particular tissue type. It does not mean that the organoid necessarily has to be genetically and phenotypically identical (or thereabouts) to the corresponding in vivo tissue cell type. However, in some aspects, the organoids used in the assay comprise cells that are genetically and phenotypically stable relative to the in vivo cell or cells that the organoid was derived from. By genetically and phenotypically stable, it is meant that there is no genetic manipulation involved, only a minimum number of mutations (i.e. close to the normal number of mutations that would be expected in in vivo cells, for example during replication and DNA synthesis).

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

As used herein, the phrase “tumor-exposed natural killer cells” can refer to NK cells that are distinct from normal (or healthy) NK cells in that they have either: a reduced cancer cell-killing property or increased ability to promote the growth, survival, or metastatic properties of cancer cells. In some aspects, tumor-exposed natural killer cells can be identified by their function or activity. In some aspects, tumor-exposed natural killer cells can be distinguished from normal NK cells based on molecular correlates of these functional or activity differences.

Cancer is the second leading cause of death in the United States and 90% of deaths occur when the cancer has spread through a body to form new tumors in distant organs, a process termed metastasis. Metastasis is poorly understood at the molecular level, few current or pipeline drugs target the biological processes driving metastasis, and existing therapies are largely ineffective for patients at metastatic stages. The limited understanding derives from the fact that metastasis is poorly modeled in traditional cell culture assays. In particular, described herein are experimental model systems that capture the interactions between cancer cells and the innate immune system in physiological settings that recapitulate important features of the invasion, dissemination, and early growth of metastatic cancer cells.

The compositions and methods disclosed herein provides a solution to the problem of modeling the interactions of natural killer (NK) cells with cancer cells in 3D culture models of cancer invasion and metastatic colonization. NK cells have a major role in the immunosurveillance of metastatic cancers (e.g. metastatic breast cancer), but little is known about the underlying mechanisms and important temporal events of NK cell immunosurveillance during metastasis. To this end, mouse tumors and mouse NK cells from the spleens of healthy donors, NK cells from the spleens of tumor bearing mice, or with NK cells recovered from their infiltrating location with the primary tumor can be used in the methods disclosed herein. While the proteins of the immune system are among the most evolutionarily divergent between mice and humans, a platform for testing immunomodulatory therapeutics based on human cells would be beneficial. The biological phenomena described herein can be recapitulated using simple 3D co-culture assays of a human cancer cell lines and human NK cells (e.g. normal or tumor-exposed natural killer cells). For example, the biological phenomena described herein can be recapitulated using simple 3D co-culture assays of a human breast cancer cell line (MCF-7) and a human NK cell line (NK-92). Further disclosed herein are (1) protocols to scale up the cell line based model for multiwell plates and high-content screening, in a reduced complexity assay with ECM, (2) adaptation of the 3D culture assay for patient derived xenograft (PDX—these are human tumors grown in mice) for use with NK cells, and (3) a fully human primary cell assay in which either fresh human tumor tissue or PDX tumor tissue that is combined with fresh primary human NK cells recovered from blood donations. The combination of these NK cell coculture specific assays with advances in automation and image analysis allows rapid, quantitative assessment of the efficacy of individual and combinations of conventional, targeted, biological, and immune-modulatory therapies in physiologically relevant settings.

Disclosed herein are methods that capture the dynamic interactions between innate immune cells (for example, natural killer (NK) cells) and cancer cells in the context of a three dimensional (3D) tissue environment. Disclosed herein are methods that use tumors from mouse or human models, patient derived xenografts (e.g. PDX cancer cells that are human, host cells are mouse), and for cancer cell lines.

The conditions for isolation, culture, and analysis of these NK-cancer cell co-cultures are described herein in a variety of formats to permit careful examination of a couple of experimental conditions as well as high throughput screening through a library of small molecule or biological compounds. Also disclosed herein are protocols to isolate and use NK cells from: the spleens of healthy subjects, the spleens of subjects bearing tumors, within a subject's tumors, and from a subject's blood. The conditions for expansion of mouse or human NK cells with mouse or human IL-2 or IL-15 can also been validated. Disclosed are 3D culture models of primary tumor invasion and dissemination (briefly, tumor organoids are embedded with NK cells (NK:tumor cells) gels of collagen I) and for metastatic colonization of distant organs (briefly, cancer cell clusters are embedded with NK cells in a basement membrane like gel). A point of distinction is the step of co-culturing with innate immune cells. The Examples provided herein describe a version of the assay.

The co-culture assays described herein can be used to demonstrate: (1) NK cells selectively kill the most metastatic cancer cells, (2) the killing can be due to direct cytotoxicity and involves caspase function, (3) NK mediated killing can be increased if antibodies selective for cell surface markers on the cancer cells are including, a phenomenon known as antibody-dependent cellular cytotoxicity, (4) cancer cells can induce NK cells to change their molecular phenotype and instead promote metastasis, (5) the molecular changes in this alternate phenotype and identified specific cell-surface receptors and ligands, and (6) that interference with these targets blocks NK cell reprogramming and leads to elimination of metastatic outgrowths.

Disclosed herein are method of using fresh human cell co-culture as a platform to evaluate immunomodulatory therapeutic concepts (small molecule or biological), as single agents and in combination. The plating, dosing, imaging, and image analysis of these assays can be automated and adapted to high throughput testing and analysis and thus can rapidly evaluate a series of concepts, with tissue from the same tumor. The methods described herein can be modified for use with other innate or adaptive immune cell populations e.g., macrophages and a variety of cancer types, including, but not limited to breast, prostate, pancreas, and lung tumor organoid monocultures.

Compositions

Disclosed herein are composition comprising an organoid/natural killer (NK) cell co-culture. In some aspects, the organoid/NK cell co-culture can comprise one or more tumor organoids with one or more NK cells embedded in an extracellular matrix.

In some aspects, the organoid can be a tumor organoid. In some aspects, the organoid can be a mammalian organoid (i.e., they are derived from cells taken from a mammal). In some aspects, the organoid can be a human organoid. In some aspects, tumor organoid can be any tumor. In some aspects, the tumor organoid can be a mammary tumor. In some aspects, the one or more organoids can be mammary tumor organoids. In some aspects, the one or more organoids can be prostate, lung, liver, or neuroblastoma tumor organoids. In some aspects, the one or more organoids can be a solid tumor organoid.

In some aspects, the one or more organoids can be generated from primary human cells.

In some aspects, the tumor organoids can be generated from an isolated primary tumor from a subject or patient. In some aspects, the tumor organoids can be generated via mechanical disruption and/or enzymatic digestion.

In some aspects, the NK cells can be from a subject or patient. In some aspects, the NK cells can be from the same subject or patient as the primary human cells that are used to generate the organoid. In some aspects, the NK cells can be from a different subject or patient as the primary human cells that are used to generate the organoid. In some aspects, the NK cells can be healthy NK cells. In some aspects, the NK cells can be tumor exposed NK cells. In some aspects, the tumor exposed NK cells can lack the ability or have a reduced ability to kill tumor cells. In some aspects, the tumor exposed NK cells have a reduced cancer cell-killing property or an increased ability to promote the growth, survival, or metastatic properties of cancer cells. In some aspects, the NK cells can be a mixture of healthy NK cells and tumor exposed NK cells.

In some aspects, the one or more tumor organoids can comprise one or more natural killer cells with Klrg1, TIGIT, or Lag3 present on its surface.

Methods of Screening

Disclosed herein are assays and methods for use in drug screening, for example, for screening a library of potential drugs.

Disclosed herein are method of identifying a drug candidate for treating cancer metastasis. In some aspects, the methods can comprise culturing an embedded organoid/natural killer (NK) cell co-culture with a drug candidate; and measuring one or more metastatic properties of the embedded organoid or tumor-killing potential or tumor-promoting potential of the NK cells in the co-culture. In some aspects, a change in the one or more metastatic properties or an increase in the tumor killing tumor potential of the NK cells or a decrease in the tumor promoting potential in the NK cells identifies a drug candidate that is capable of treating cancer metastasis.

Disclosed herein are methods of identifying a drug candidate that inhibits natural killer cell activity. In some aspects, the methods can comprise: culturing an embedded organoid/NK cell co-culture with a drug candidate; and measuring one or more metastatic properties of the embedded organoid or tumor-killing potential or tumor-promoting potential of the NK cells in the co-culture. In some aspects, a change in the one or more metastatic properties or an increase in the tumor killing tumor potential of the NK cells or a decrease in the tumor promoting potential in the NK cells identifies a drug candidate capable of inhibiting natural killer cell activity. In some aspects, the activity of the NK cell can be measured by molecular methods, for example, using qRT-PCR. In some aspects, the NK cell activity can be measured by determining the expression level of one or more inhibitory or activating genes.

Disclosed herein are methods of identifying a cancer patient's responsiveness to an anticancer drug candidate. In some aspects, the methods can comprise: culturing an embedded organoid/NK cell co-culture with a drug candidate; and measuring one or more metastatic properties of the embedded organoid or tumor-killing potential or tumor-promoting potential of the NK cells in the co-culture. In some aspects, wherein a change in the one or more metastatic properties or an increase in the tumor killing tumor potential of the NK cells or a decrease in the tumor promoting potential in the NK cells identifies an anticancer drug that the subject is responsive to.

Disclosed herein are methods of determining the molecular phenotype of a NK cell in sample from a subject. In some aspects, the subject has cancer. In some aspects, the sample can be a blood sample. In some aspects, the sample from be a biopsy sample. In some aspects, the methods can compare the molecular phenotype of a NK cell from two different sample types from the same subject or from the same sample type from the same subject to determine the molecular phenotype of the NK cells to determine the degree of change or amount of change of an active vs. resting molecular phenotypes. In some aspects, said information can be used to inform drug selection. In some aspects, the methods can be performed using PCR or sequencing. In some aspects, the active molecular NK phenotype can correspond to a healthy NK molecular phenotype. In some aspects, the resting molecular NK phenotype can correspond to a reduced cancer cell-killing property or increased ability to promote the growth, survival, and/or metastatic properties of cancer cells. In some aspects, the molecular phenotype of a NK cell can be determined in a sample directly from a subject. In some aspects, the molecular phenotype of a NK cell can be determined after the NK cell has been co-cultured with an organoid as described herein.

Disclosed herein are methods identifying an antibody that binds to a specific tumor antigen. In some aspects, the methods can comprise: culturing an embedded organoid/natural killer (NK) cell co-culture with an antibody; and measuring one or more metastatic properties of the embedded organoid or tumor-killing potential or tumor-promoting potential of the NK cells in the co-culture. In some aspects, a change in the one or more metastatic properties or an increase in the tumor killing tumor potential of the NK cells or a decrease in the tumor promoting potential in the NK cells means that the antibody binds to the specific tumor antigen.

Disclosed herein are methods identifying a drug candidate for treating cancer metastasis. In some aspects, the methods can comprise: culturing an embedded organoid/natural killer (NK) cell co-culture with a drug candidate; and measuring one or more metastatic properties of the embedded organoid or tumor-killing potential or tumor-promoting potential of the NK cells in the co-culture. In some aspects, a change in the one or more metastatic properties or an increase in the tumor killing tumor potential of the NK cells or a decrease in the tumor promoting potential in the NK cells means that the antibody binds to the specific tumor antigen.

Disclosed herein are methods of identifying a drug candidate for treating cancer metastasis. In some aspects, the methods can comprise: culturing an embedded organoid/natural killer (NK) cell co-culture with a drug candidate; and measuring one or more metastatic properties of the embedded organoid or tumor-killing potential or tumor-promoting potential of the NK cells in the co-culture. In some aspects, the one or more tumor organoids comprise one or more natural killer cells with Klrg1, TIGIT, or Lag3 present on its surface. In some aspects, the one or more tumor organoids can be in contact with or in co-culture with one or more natural killer cells with Klrg1, TIGIT, or Lag3 present on its surface. In some aspects, a change in the one or more metastatic properties or an increase in the tumor killing tumor potential of the NK cells or a decrease in the tumor promoting potential in the NK cells means that the drug candidate inhibits binds to Klrg1, TIGIT, or Lag3 present on the surface of the one or more natural killer cells. In some aspects, the change in the one or more metastatic properties or an increase in the tumor killing tumor potential of the NK cells or a decrease in the tumor promoting potential in the NK cells indicates that the subject is responsive to treatment with the drug.

Disclosed herein are methods of identifying one or more biomarkers of cancer metastasis. In some aspects, the methods can include culturing an embedded organoid/natural killer (NK) cell co-culture, wherein the NK cell is a normal NK cell and separately culturing an embedded organoid/natural killer (NK) cell co-culture, wherein the NK cell is a tumor exposed NK cells and identifying biomarkers that are differentially expressed or present between the two co-cultures. In some aspects, the methods can further comprise analyzing the NK cells exposed to the tumor. In some aspects, the methods can predict or identify one or more biomarkers of a tumor exposed NK cell.

In some aspects, the methods disclosed herein can further comprise culturing one or more tumor organoids with one or more natural killer (NK) cells to provide a organoid/NK cell co-culture and embedding the organoid/NK cell co-culture in an extracellular matrix to provide an embedded organoid/NK cell co-culture prior to the step of culturing an embedded organoid/natural killer (NK) cell co-culture with a drug candidate.

In some aspects, the drug candidate can be an immune receptor inhibitor or an immune receptor activator. In some aspects, the drug candidate can be an antibody.

In some aspects, the one or more tumor organoids can comprise one or more natural killer cells with Klrg1, TIGIT, or Lag3 present on its surface.

In some aspects, the change in the one or more metastatic properties or the increase in the tumor killing tumor potential of the NK cells or the decrease in the tumor promoting potential in the NK cells can be determined by comparison to a reference or control organoid or NK cell.

In some aspects, the one or more metastatic properties measured can be metabolic activity, cell proliferation, apoptosis, cancer invasion, or molecular or cellular correlates of metastatic ability. In some aspects, metabolic activity can be measured using a MTT assay. In some aspects, cell proliferation can be measured using antibody staining for, for example, Ki67 or pHH3. In some aspects, apoptosis or cell death can be measured by assessing caspase activity, propidium iodide or ethidium homodimer binding. In some aspects, cancer invasion can be determined using shape analysis on images. In some aspects, molecular and cellular correlates of metastatic ability can be determined. In some aspects, activity of the NK cells can be measured by cellular or molecular means. In some aspects, the molecular means can be qRT-PCR. In some aspects, the change in one or more metastatic properties or tumor killing tumor potential of the NK cells or the change in the tumor promoting potential in the NK cells can be determined or measured by assessing colongy formation. In some aspects, the change in one or more metastatic properties or tumor killing tumor potential of the NK cells or the change in the tumor promoting potential in the NK cells can be determined by detecting one or more NK cell surface markers.

In some aspects, the method can be a high-throughput screening assay. For example, in some aspects, the organoids can be cultured in an array format, for example in multiwell plates, such as 96 well plates or 384 well plates.

In some aspects, the organoids in the drug screen, for example in the array, can be derived from one individual subject or patient. In some aspects, the organoids in the drug screen, for example in the array, can be derived from different patients. In some aspects, the drug screen, for example the array, comprises organoids derived from one or more diseased patients in addition to organoids derived from one or more healthy controls.

Libraries of molecules can be used to identify a molecule that affects the organoids. For example, libraries that comprise antibody fragment libraries, peptide phage display libraries, peptide libraries (e.g. LOPAP, Sigma Aldrich), lipid libraries (BioMol), synthetic compound libraries (e.g. LOP AC, Sigma Aldrich) natural compound libraries (Specs, TimTec) or small molecule libraries can be used. Furthermore, genetic libraries can be used that induce or repress the expression of one of more genes in the progeny of the stem cells. Such genetic libraries include cDNA libraries, antisense libraries, and siRNA or other non-coding RNA libraries. In the methods disclosed herein, the embedded organoid/natural killer (NK) cell co-culture can be exposed to multiple concentrations of a test agent for a certain period of time. At the end of the exposure period, the embedded organoid/natural killer (NK) cell co-cultures can evaluated. The term “affecting” is used to cover any change in the organoid or NK cell, including, but not limited to, a reduction in, or loss of, proliferation, a morphological change, and cell death. In some aspects, the organoids can be used in the assay to test libraries of chemicals, antibodies, natural product (plant extracts), etc. for suitability for use as drugs, cosmetics and/or preventative medicines. For instance, in some aspects, a cell biopsy from a patient of interest, such as tumor cells from a cancer patient, can be cultured using culture media and methods described herein and then treated with a drug or a screening library. It is then possible to determine which drugs effectively interact with natural killer cells and cancer cells. This can allow specific patient responsiveness to a particular drug to be tested thus allowing treatment to be tailored to a specific patient. Thus, this allows a personalized medicine approach.

The added advantage of using the organoids for identifying drugs as described herein is that it is also possible to screen normal organoids (organoids derived from healthy tissue) to determine which drugs and compounds have minimal effect on healthy tissue. This can allow for screening for drugs with minimal off-target activity or unwanted side-effects.

In some aspects, the methods are for testing the effect of novel drugs to reduce or halt cancer metastasis.

In some aspects, the methods or assays described herein can use the disclosed organoids to test effect of novel drugs to affect immune receptor based recognition of cancer cells by natural killer cells. In some aspects, the methods can be used to test the efficacy of candidate drugs or agents that target immune inhibitory or activating receptors.

In some aspects, the NK cell activity in the assay or method can be increased by adding antibodies that recognize the cancer cells. In some aspects, the methods disclosed herein can be used to test the efficacy (and selectivity) of antibodies that target cancer cells with the purpose of recruiting immune function.

As described herein, the methods disclosed herein can be used to determine the mechanisms in which cancer cells can reprogram NK cells to promote metastasis. By identifying the immune receptors that mediate this reprograming, molecular strategies can be developed to prevent the reprogramming of the NK cells and reduce or prevent metastasis. In some aspects, the methods disclosed herein can be used to evaluate the efficacy of drug candidate and agents that target the one or more molecules important in innate immune system function (e.g., Klrg1, TIGIT, Lag3). In some aspects, the methods disclosed herein can be used to screen small molecule or biological compounds or libraries for their activity in promoting NK-mediated cancer cell killing or preventing NK reprogramming.

In some aspects, the methods can be used for testing the effect of novel drugs for their ability to promote NK-mediated cancer cell killing or prevent NK reprogramming, for example for the treatment of cancer (e.g., breast cancer).

In some aspects, the candidate drug being tested is selected from a synthetic small molecule, protein, peptide, antibody (or derivative thereof), aptamer and nucleic acid (such as an antisense compound).

In some aspects, the methods can further comprise the step of selecting the effective drug and optionally using said drug for treatment.

The invention also provides the use of one or more organoids for drug screening, wherein the drug screening comprises using an assay according to the invention.

Use of the Methods in Personalized Medicine. In some aspects, the methods disclosed herein can use organoids that are patient derived tumor (e.g., mammary) organoids for the assessment of the individual responsiveness to certain treatment options.

In some aspects, the methods comprise contact or co-culturing the one or more organoids with one or more drug candidates, for example for use in personalized medicine.

In some aspects, the methods disclosed herein can be for use in personalized medicine, for example to test individual patient response to drugs for the disease or affliction of interest.

In some aspects, the methods disclosed herein for use in personalized medicine can be used to test individual patient response to drugs wherein the disease of interest is cancer (e.g., breast cancer), and wherein the assay comprises contacting or co-culturing the one or more organoids derived from a patient with a compound; wherein an reduction in colony formation indicates that the patient is responsive to treatment with the drug.

Thus, the methods provide the use of one or more organoids for the assessment of the responsiveness to a particular treatment option, wherein the assessment comprises use of an assay as described herein and wherein a reduction in colongy formation of the organoid is indicative of successful treatment.

Also disclosed herein are methods of treating a disease or affliction, the methods comprising using the assay described herein for identifying a drug for the disease or an affliction that a patient is responsive to, and treating the patient with said drug. In some aspects, the drug can be any known or putative drug for treating a disease or affliction associated promoting NK-mediated cancer cell killing or preventing NK reprogramming or DNA methyltransferases inhibitors or anti-TIGIT antibodies or anti-KLRG1 antibodies. In some aspects, the drug can a known or putative drug for cancer or breast cancer or reducing metastatic potential.

In some aspects, computer- or robot-assisted culturing and data collection methods can be employed to increase the throughput of the screen.

In some aspects, the organoid can be obtained from a patient biopsy. In some aspects, the candidate drug that causes a desired effect on the organoid can be administered to said patient.

### EXAMPLES

### Example 1: Cancer Cells Educate Natural Killer Cells to a Metastasis Promoting Cell State

Described herein are findings demonstrating that natural killer (NK) cells can be reprogrammed by breast cancer cells to promote metastasis. Reprogramming can be blocked by targeting NK cell inhibitory receptors TIGIT or KLRG1 or inhibiting DNA methyltransferases. The methods described herein can be used to identify therapeutic agents that prevent or treat metastasis.

Natural killer (NK) cells have potent anti-tumor and anti-metastatic activity. It is incompletely understood how cancer cells escape NK cell surveillance. Using ex vivo and in vivo models of metastasis, it was established that keratin-14-positive breast cancer cells are vulnerable to NK cells. Then, it was discovered that exposure to cancer cells causes NK cells to lose their cytotoxic ability and promote metastatic outgrowth. Gene expression comparisons revealed that healthy NK (hNK) cells have an active NK cell molecular phenotype while tumor-exposed (teNK) cells resemble resting NK cells. Receptor-ligand analysis between teNK cells and tumor cells revealed multiple potential targets. The results show that treatment with antibodies targeting TIGIT, antibodies targeting KLRG1, or small molecule inhibitors of DNA methyltransferases (DMNT) each reduced colony formation. Combinations of DNMTs inhibitors with anti-TIGIT or anti-KLRG1 antibodies further reduced metastatic potential. Disclosed herein are methods directed to NK-directed therapies that target these pathways can be effective in the adjuvant setting to prevent metastatic recurrence.

Natural killer (NK) cells are components of the innate immune system and have potent anti-tumor and anti-metastatic activity (Lopez-Soto A, et al. Cancer Cell. 2017; 32(2):135-54). Accordingly, breast cancer cells must overcome NK cell surveillance in order to form distant metastases. Yet, until now, it is not well understood how metastatic cancer cells escape NK cell regulation. Others have shown that breast cancer cells, through a dormant state, downregulate activating receptors to evade NK cells (Malladi S, et al. Cell. 2016; 165(1):45-60). However, until now, it was not fully understood how breast cancer cells escape NK cell mediated immunosurveillance during transit through the circulation and initial seeding of distant organs. Mechanistic studies have also been limited by the availability of appropriate models to study NK cell-cancer cell interactions in physiologically realistic 3D settings.

Breast tumors exhibit significant molecular heterogeneity, potentially explaining observed differences in metastatic potential and treatment response (Janiszewska M, et al. Nat Cell Biol. 2019; 21(7):879-88; and Marusyk A, et al. Nat Rev Cancer. 2012; 12(5):323-34). It has been demonstrated that keratin-14 (1(14) defines a subpopulation of breast cancer cells that lead collective invasion, systemic dissemination, and colonization of distant organs (Cheung K J, et al. Cell. 2013; 155(7):1639-51; Cheung K J, and Ewald A J. Science. 2016; 352(6282):167-9; and Cheung K J, et al. Proc Natl Acad Sci USA. 2016; 113(7):E854-63). As described herein, novel ex vivo co-cultures and in vivo metastasis models were used to understand the cellular interactions between NK cells and K14+ cancer cells and to elucidate the molecular mechanisms by which breast cancer cells escape NK cell immunosurveillance to establish distant metastases.

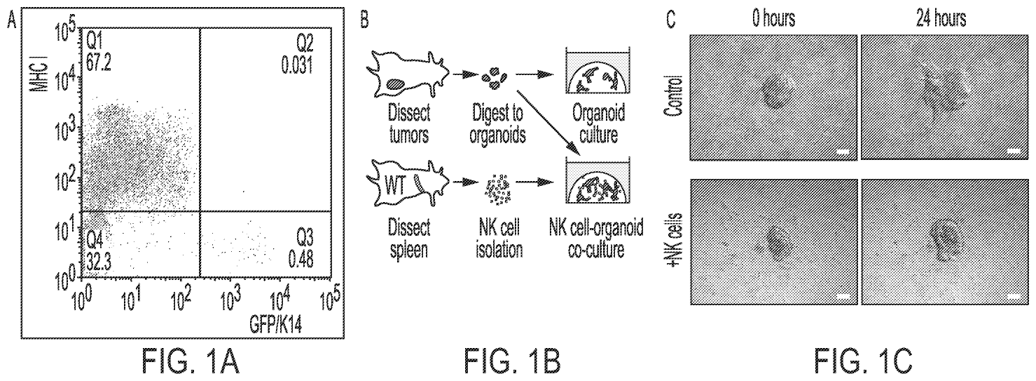

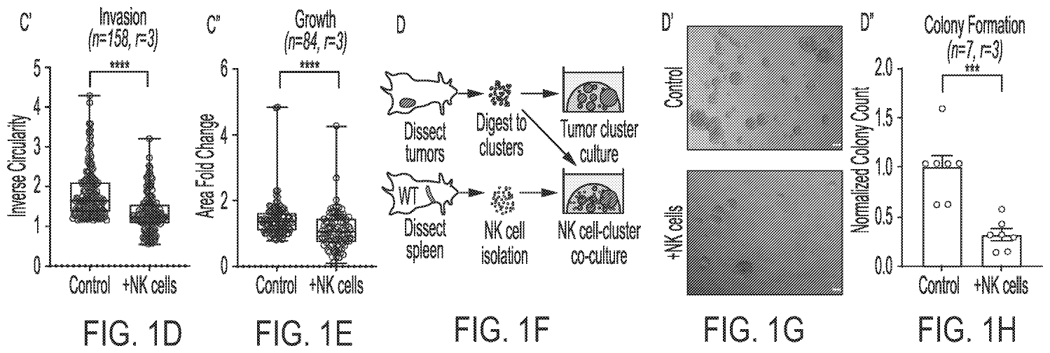

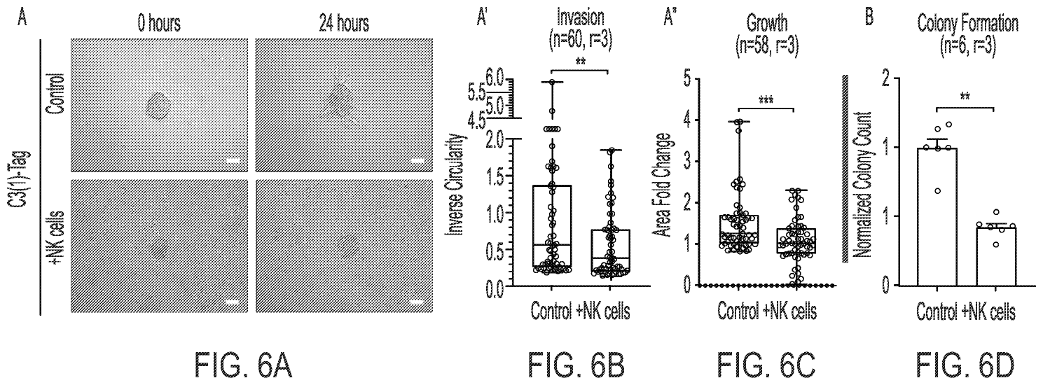

To determine how K14+ cells evade immunosurveillance, K14− and K14+ cells were isolated by fluorescence activated cell sorting (FACS) from MMTV-PyMT (Guy C T, et al. Mol Cell Biol. 1992; 12(3):954-61) tumors with a genetically encoded K14 fluorescent reporter, then stained for MHC class I molecules, which are important inhibitors of NK cell activity (Morvan M G and Lanier L L. Nat Rev Cancer. 2016; 16(1):7-19). A striking inverse relationship was observed between K14 status and MHC class I expression, suggesting that K14+ cancer cells are susceptible to NK cell mediated cytotoxicity (FIG. 1A). A NK cell-tumor organoid ex vivo co-culture system was then developed (FIG. 1B). Briefly, freshly isolated NK cells from the spleens of healthy congenic mice were activated with IL-2 or IL-15 and then embedded with organoids derived from mammary tumors in 3D collagen I gels. A time-lapse differential interface contrast (DIC) microscopy was used to determine the impact of healthy NK (hNK) cells on invasion in both the MMTV-PyMT and C3(1)-Tag (Maroulakou I G, et al. Proc Natl Acad Sci USA. 1994; 91(23):11236-40) models of murine mammary tumors (FIG. 1C, FIG. 6A). Co-culture with hNK cells reduced tumor organoid invasion by ˜40% in PyMT organoids and ˜50% in C3(1)-Tag organoids within the first 24 hours of culture (FIGS. 1D-E, FIGS. 6B-C). Organoid growth was decreased 20-30% in both models suggesting that hNK cells preferentially target cancer cells involved in collective invasion (FIG. 1E, 6C). Next, the effect of hNK cells on distant organ seeding was modeled using a colony forming assay in which 2-3 cell clusters are embedded in Matrigel (FIG. 1D, as in (Cheung K J, et al. Proc Natl Acad Sci USA. 2016; 113(7):E854-63; and Padmanaban V, et al. Nature. 2019; 573(7774):439-44). Co-culture with hNK cells reduced colony formation by ˜70% in clusters derived from MMTV-PyMT mice and 60% in clusters derived from C3(1)-Tag mice (FIGS. 1G-H, FIG. 6D).

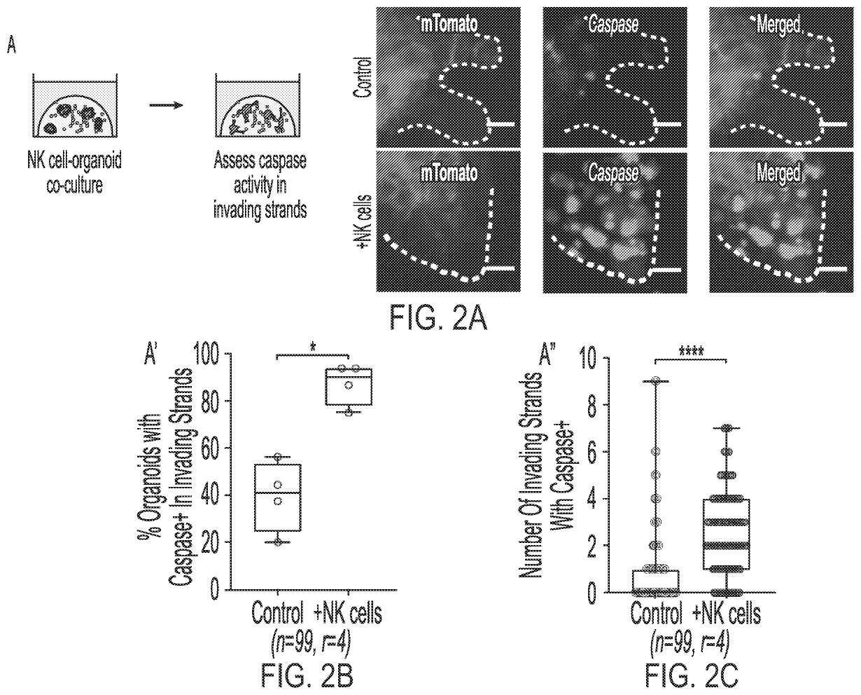

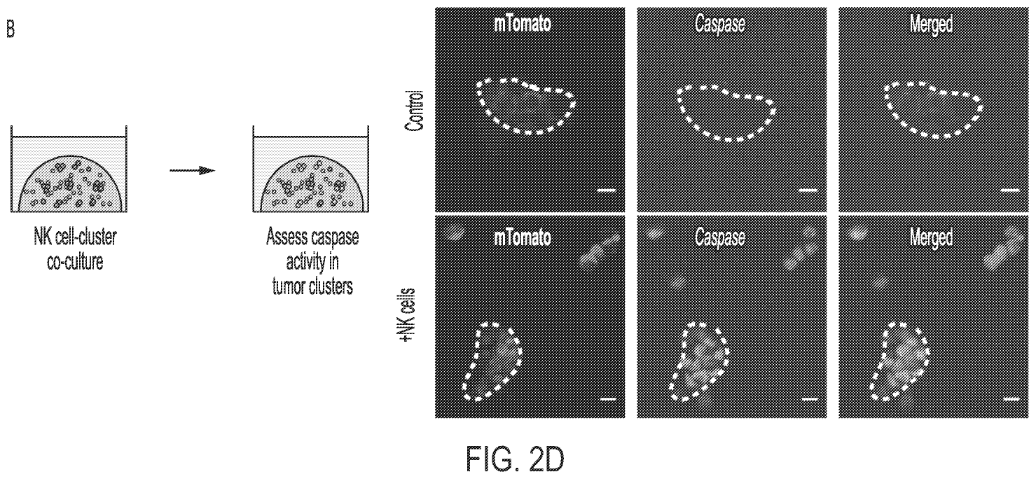

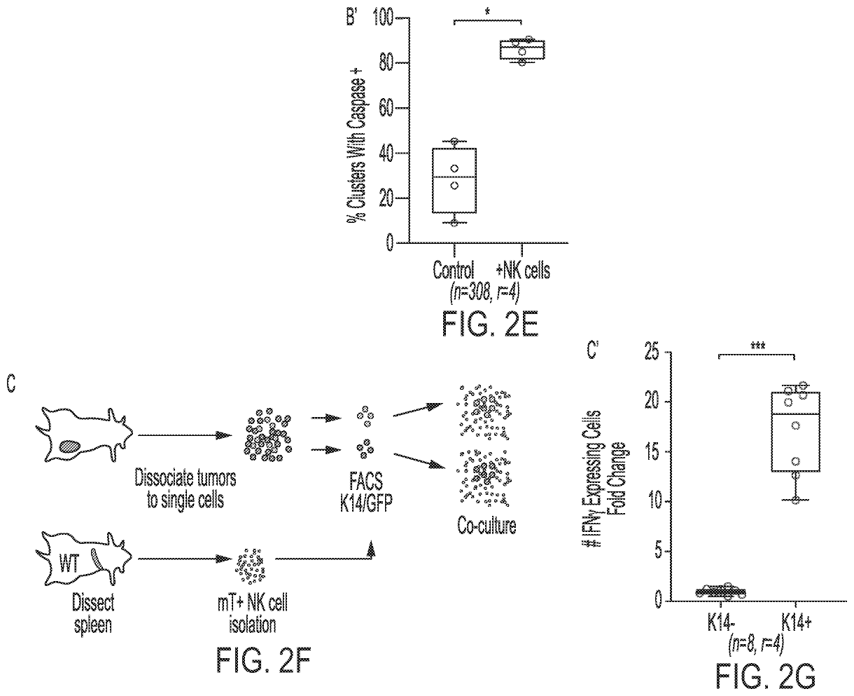

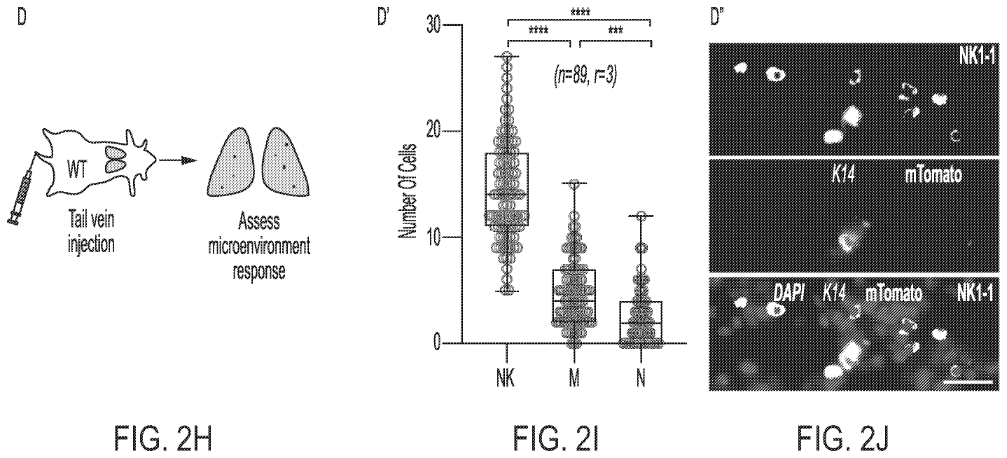

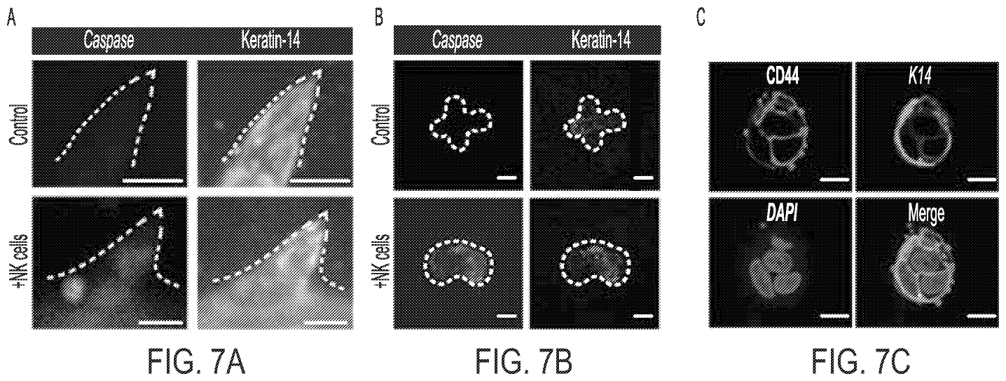

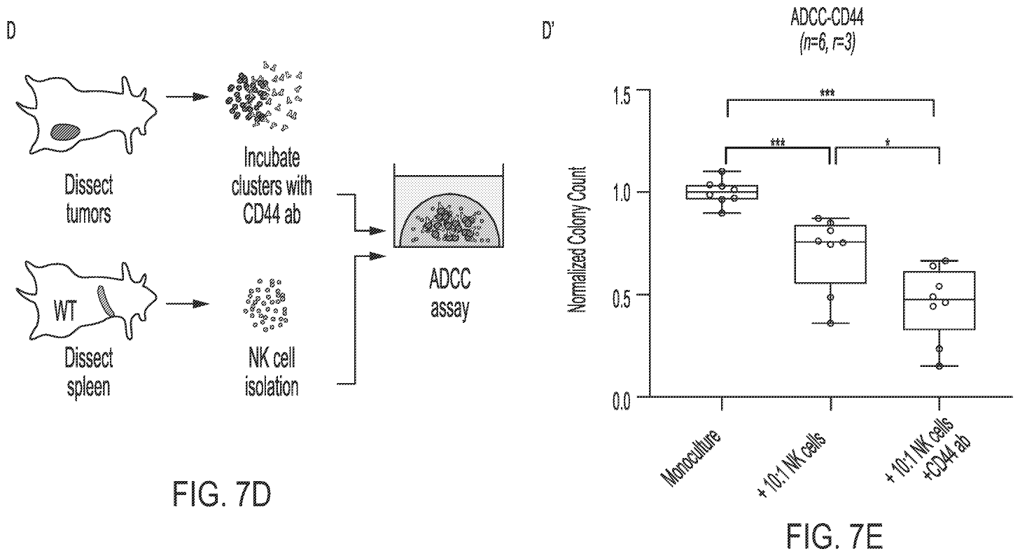

Next, experiments were carried out to identify the cellular mechanism by which hNK cells limit invasion and colony formation. A caspase-3/7 biosensor enabled real-time analysis of apoptosis dynamics during interactions between hNK cells and tumor organoids. Caspase 3/7 activity within invasive cancer cells when co-cultured with hNK cells during the first 12-24 hours. In the organoid invasion assays, ˜43% of mono-culture organoids and ˜85% of hNK cell co-cultured organoids exhibited caspase activity, with multiple caspase biosensor+invasion strands observed per organoid (FIGS. 2A-C). Consistent with their MHC class I-negative status, caspase activity localized to K14+ cancer cells within the invasive strand (FIG. 7A). Next, apoptosis was examined at 6-8 hours in the colony forming assay and caspase+ cells were observed in ˜25% of mono-culture and ˜85% of co-culture clusters (FIGS. 2D-E). As before, apoptosis was enriched in K14+ cells within the clusters (FIG. 7B). Together, these data show that hNK cells limit invasion and colony formation through direct cytotoxicity of K14+ cells. As molecular validation of this concept, hNK cells were cultured with either FACS-sorted K14+ or K14− cancer cells and assayed for interferon-gamma expression (FIG. 2F). A >17-fold increase of interferon-gamma producing cells among hNK cells co-cultured with K14+ cells, relative to K14− co-culture was observed (FIG. 2G). This result provides direct evidence that hNK cells preferentially respond to K14+ cells.

The specificity of NK cells against their target cells can be increased through antibody-dependent cell-mediated cytotoxicity (ADCC), in which NK cell receptors bind to antibodies against specific tumor antigens (Clynes R A, et al. Nat Med. 2000; 6(4):443-6). Therefore, it was tested whether hNK cell targeting of K14+ cells could be enhanced with ADCC. Prior RNA-seq analysis of K14+ cells revealed high expression of the cell surface receptor CD44 relative to K14− cells and immunofluorescence revealed double positive (K14+; CD44+) cancer cell clusters (FIG. 7C). The co-culture assay was modified to pre-treat clusters with anti-CD44 antibody prior to co-culture with an intermediate concentration of hNK cells (FIG. 7D). The combination of CD44 pretreatment and hNK cell co-culture further decreased colony formation relative to NK cell co-culture alone (FIG. 7E). Taken together, these results provide functional evidence that hNK cells are selectively targeting K14+ cells and that their efficacy can be increased by ADCC-based strategies.

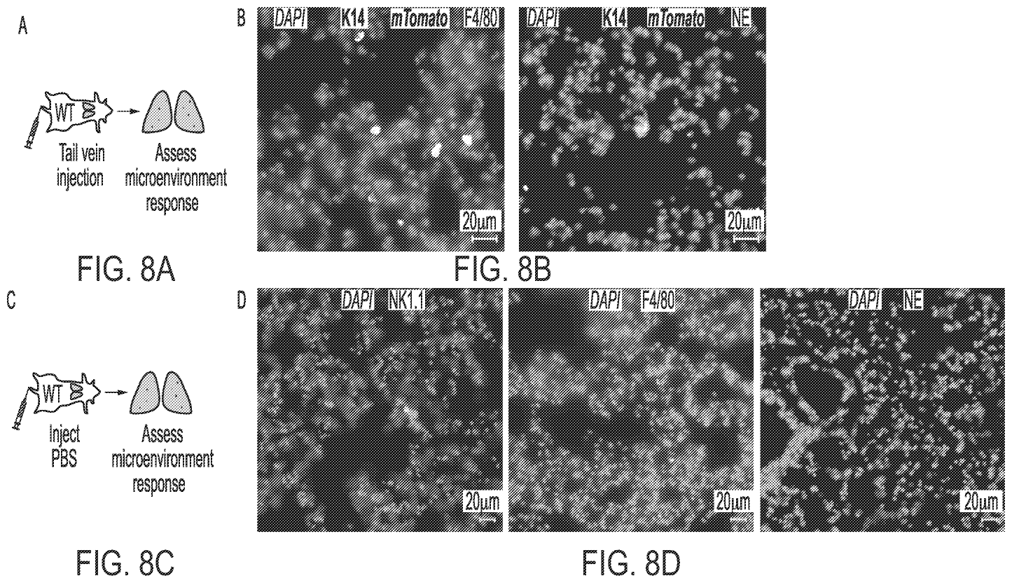

Having demonstrated that hNK cells can reduce colony formation ex vivo by apoptotic targeting of K14+ cells, the acute response of hNK cells to metastatic seeds in vivo was characterized. K14-GFP+; mTomato+ cancer cell clusters were injected into immunocompetent mice and assayed NK cell, neutrophil, and macrophage abundance at 6 hours (FIG. 2H). The most frequent responders to K14+ tumor cell cluster were NK cells, while few were observed in PBS-treated lungs (FIG. 2I-J, FIGS. 8A-D). NK cells are therefore positioned to mediate the early response to the arrival of metastatic cancer cells in distant organs.

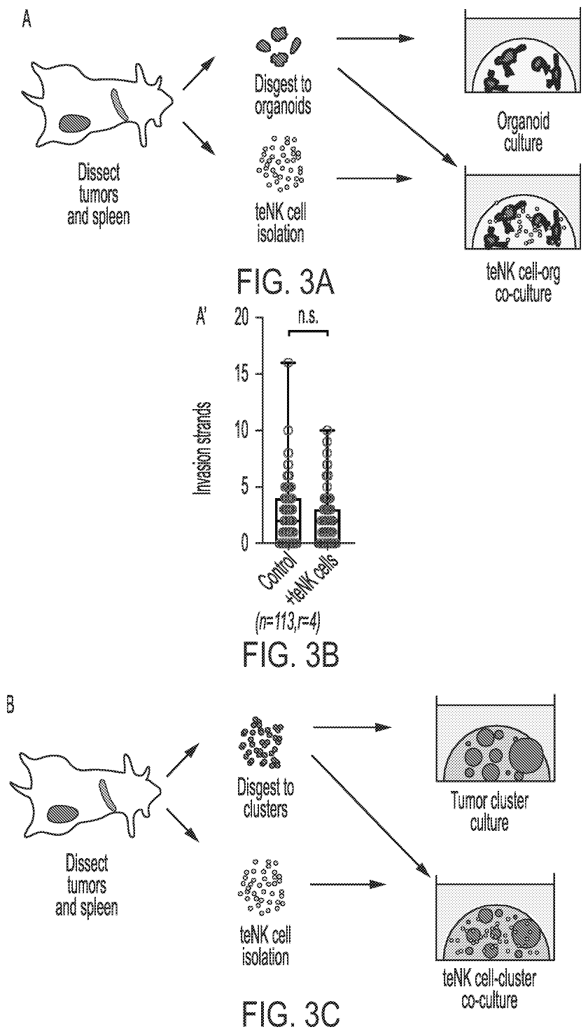

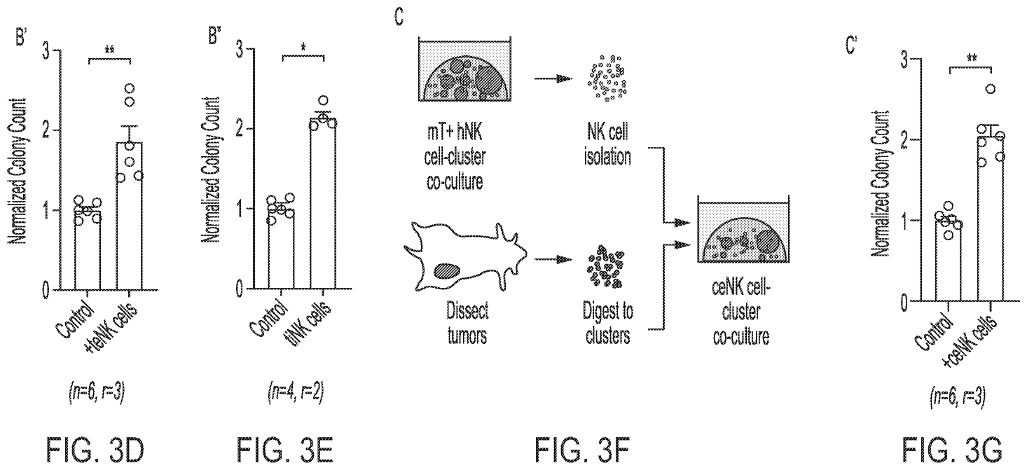

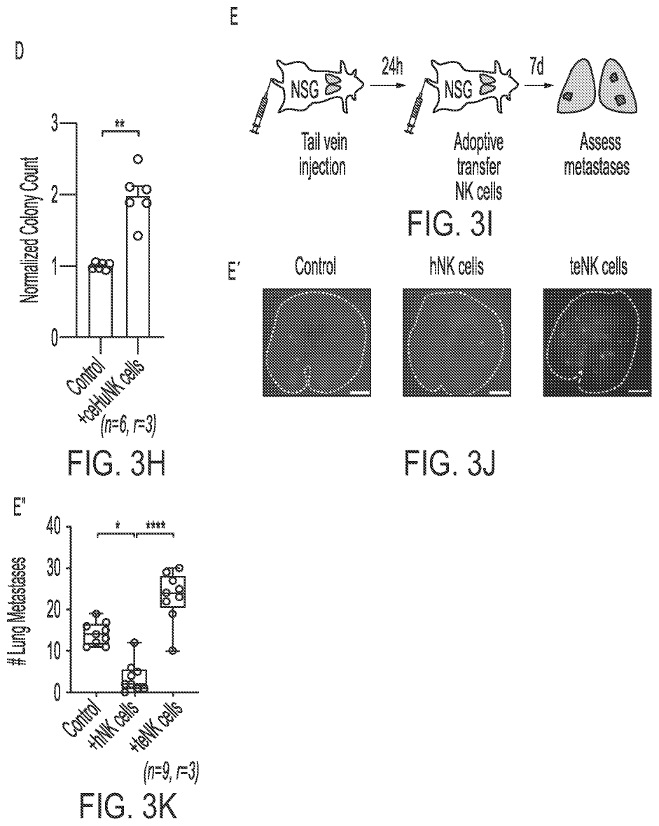

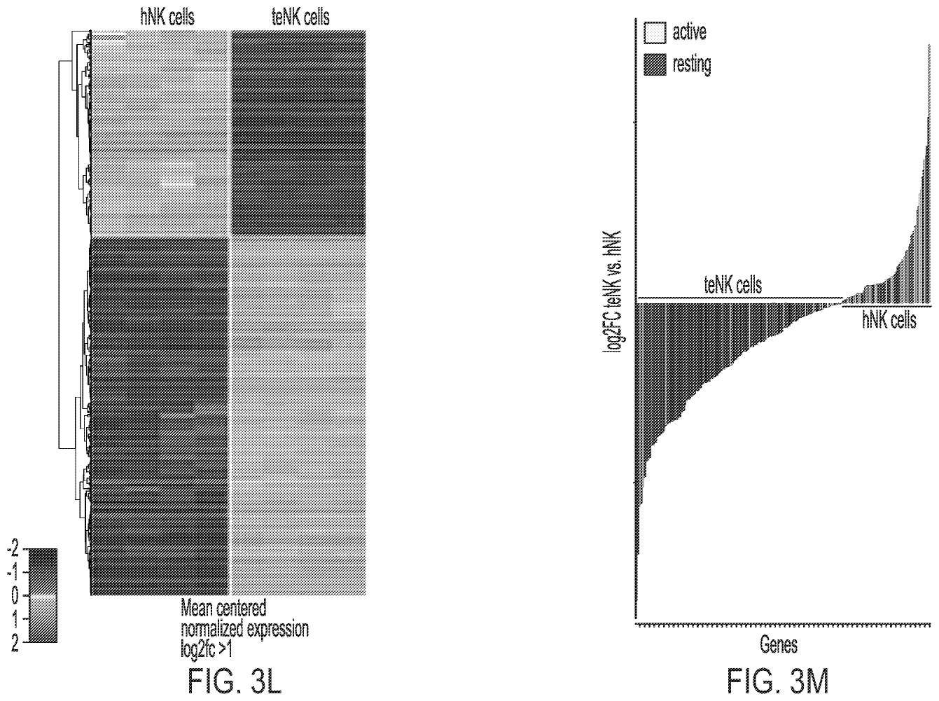

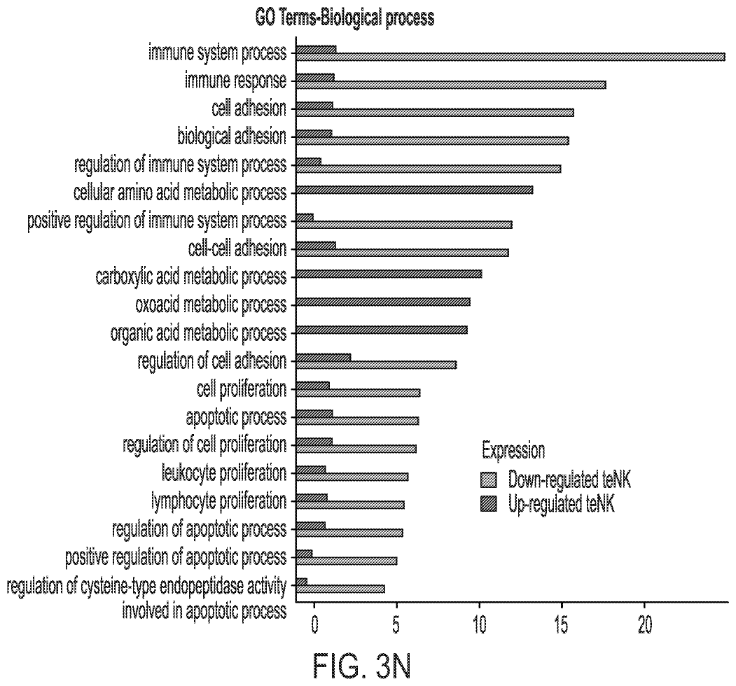

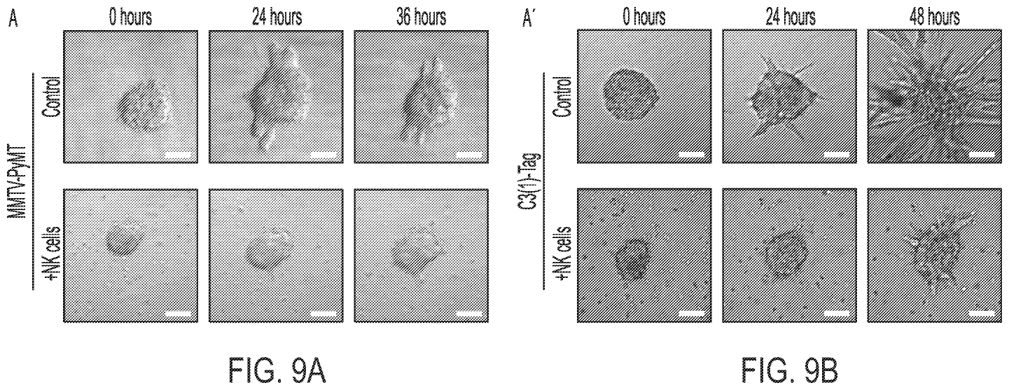

It was also observed that both invading organoids and growing colonies were able to partially overcome hNK cell cytotoxicity by 36-48 hours of culture (FIG. 9A-B). It was assessed whether the cancer cells were reprogramming NK cell differentiation. NK cells were isolated from the spleens of MMTV-PyMT tumor-bearing mice and placed in co-culture with tumor organoids (FIG. 3A). Tumor-exposed NK (teNK) cells did not limit organoid invasion (FIG. 3B). Next, teNK cells were placed in co-culture with tumor cell clusters (FIG. 3C). Surprisingly, they promoted colony formation by almost 2-fold (FIG. 3D). teNKs from the spleen were used because they can readily be isolated in large numbers. To validate that their behavior was consistent with that of NK cells in contact with tumor cells in vivo, tumor infiltrating NK (tiNK) cells were isolated from primary tumors and co-cultures were generated with a similar schema. It was observed that tiNK cells promoted colony formation to a similar degree as teNK cells (FIG. 3E). These data suggest that cancer cells can reprogram NK cells to support metastatic progression. Next, tumor education of NK cells were modeled ex vivo through exposure to tumor clusters. Fluorescently labeled hNK cells from control FVB/n mice were first co-cultured with cell clusters derived from MMT-PyMT tumors for 48 hours, processed to single cells, used FACS to isolate culture-educated NK (ceNK) cells, then co-cultured these ceNKs with new tumor cell clusters (FIG. 3F). Strikingly, co-culture with ceNK increased colony formation by 2-fold, similar to the effect of freshly isolated teNK cells (FIG. 3G). To functionally validate the murine findings in human models and assess whether human NK cells could be educated, a parallel schema was used to FIG. 3F by culture educating human NK-92 cells with MCF-7 cell clusters. It was found that culture-educated human NK (ceHuNK) cells can also promote colony formation (FIG. 3H). To test whether teNK cells promote colony formation in vivo, a tail vein assay was performed with fluorescently labeled MMTV-PyMT organoids, followed by adoptive transfer of teNK cells or hNK cells (FIG. 3I). Consistent with the results of the ex vivo colony forming assay, hNK cells significantly reduced the number of macrometastases and teNK cells significantly increased the number of macrometastases relative to hNK cells (FIGS. 3J-K). These results demonstrate that hNK cells can limit metastasis formation and that tumor education can co-opt NK cells to promote metastasis.

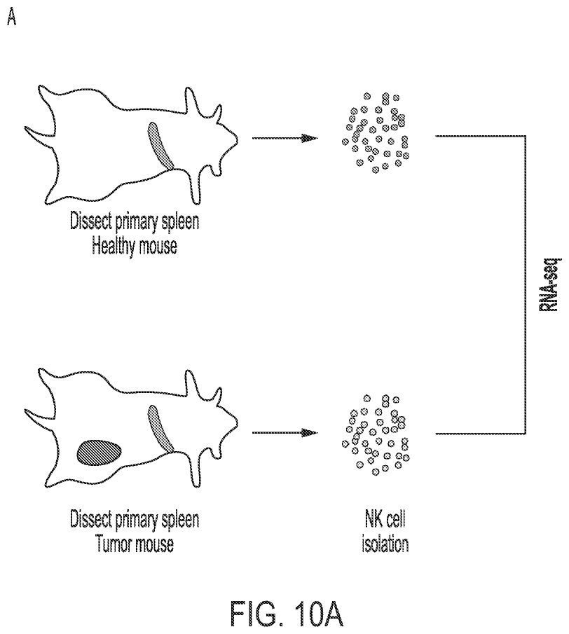

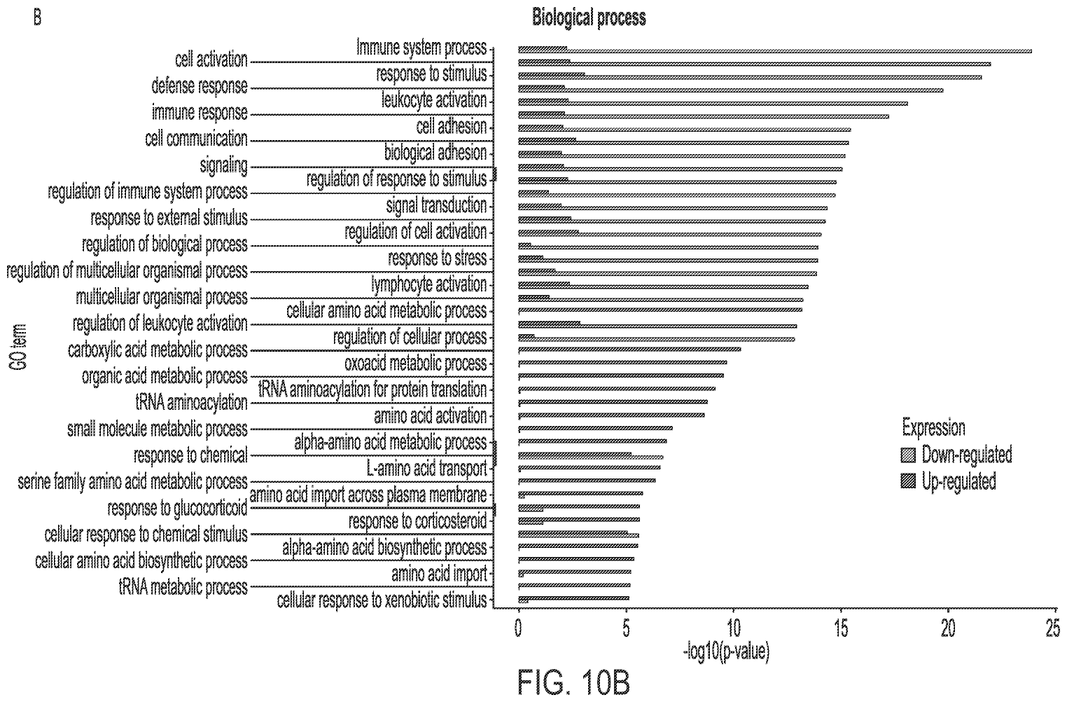

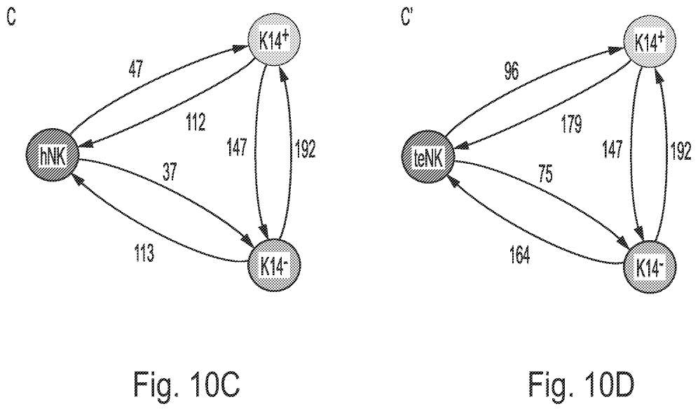

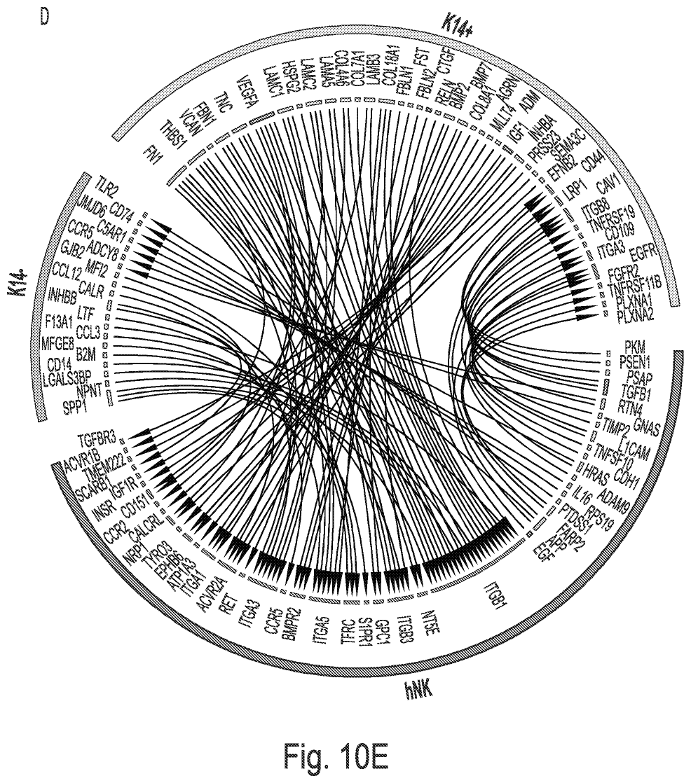

To identify the molecular mechanisms underlying tumor education of NK cells, RNA-seq analysis of hNK cells and teNK cells isolated from FVB/n and MMTV-PyMT mice, respectively, was performed (FIG. 10A). Thresholds of 2-fold up-regulated or down-regulated and false-discovery rate under 5% yielded 2604 differentially expressed genes between teNK cells and hNK cells (FIG. 3C), with 1574 genes increased and 1030 decreased in teNK cells relative to hNK cells (see, Chan et al. J. Cell Bio. 2020 Vol. 219 No. 9, p. 1). Next, the ImmuCC gene signatures (Chen Z, et al. Front Immunol. 2018; 9(1286)) were used and the data was deconvoluted using CIBERSORT (Newman A M, et al. Nat Methods. 2015; 12(5):453-7), a method of resolving relative fractions of cell types from complex mixtures. Using a subset of the gene signatures comprising NK cells, dendritic cells, and neutrophils, over 95% of the relative fraction of cells were identified as NK cells (Table 1). When compared to other innate immune cell types, the differential genes expressed by the teNK population corresponded to a resting NK cell phenotype (FIG. 3M, see, Chan et al. J. Cell Bio. 2020 Vol. 219 No. 9, p. 1, Supplemental Table 3). Genes upregulated by teNK cells had Gene Ontology annotations relating to the negative regulation of extrinsic apoptosis, while genes downregulated by teNK cells included those associated with the proliferation, metabolism, and activation of immune response to tumor cells, and the positive regulation of apoptotic process (FIG. 3N, FIG. 10B).

Examples of genes differentially expressed by teNK cells relative to hNK cells can include the genes identified in Chan et al. J. Cell Bio. 2020 Vol. 219 No. 9, p. 1 (Supplemental Table 2).

TABLE 1
CIBERSORT deconvolution analysis of genes expressed by hNK cells and teNK
cells using immune cell signatures from ImmuCC.

| Input | Neutrophil | NK | NK | DC | DC | P- | Pearson | |
|---|---|---|---|---|---|---|---|---|
| Sample | Cells | Resting | Active | Active | Immature | value | Correlation | RMSE |
| hNK | 0.018 | 0.492 | | 0 | 0 | 0 | 0.389 | 1.207 |
| sample 1 | | | | | | | | |
| hNK | 0.010 | 0.406 | 0.490 | 0 | 0 | 0 | 0.425 | 1.174 |
| sample 2 | | | | | | | | |
| hNK | 0.018 | 0.262 | 0.584 | 0 | 0 | 0 | 0.459 | 1.134 |
| sample 3 | | | | | | | | |
| hNK | 0.012 | 0.299 | 0.720 | 0 | 0 | 0 | 0.429 | 1.167 |
| sample 4 | | | | | | | | |
| teNK | 0 | 0.983 | 0.689 | 0.017 | 0 | 0 | 0.650 | 1.000 |
| sample 1 | | | | | | | | |
| teNK | 0 | 0.976 | 0 | 0.024 | 0 | 0 | 0.634 | 1.015 |
| sample 2 | | | | | | | | |
| teNK | 0 | 0.982 | 0 | 0.018 | 0 | 0 | 0.638 | 1.015 |
| sample 3 | | | | | | | | |
| teNK | 0 | 0.980 | 0 | 0.020 | 0 | 0 | 0.653 | 0.994 |
| sample 4 | | | | | | | | |

Examples of genes from ImmuCC used to determine active and resting NK phenotypes can include genes identified in Chan et al. J. Cell Bio. 2020 Vol. 219 No. 9, p. 1, Supplemental Table 3. The relative fraction of cells from healthy NK cell and tumor-exposed NK cell subset are consistent with gene signatures related to NK cells.

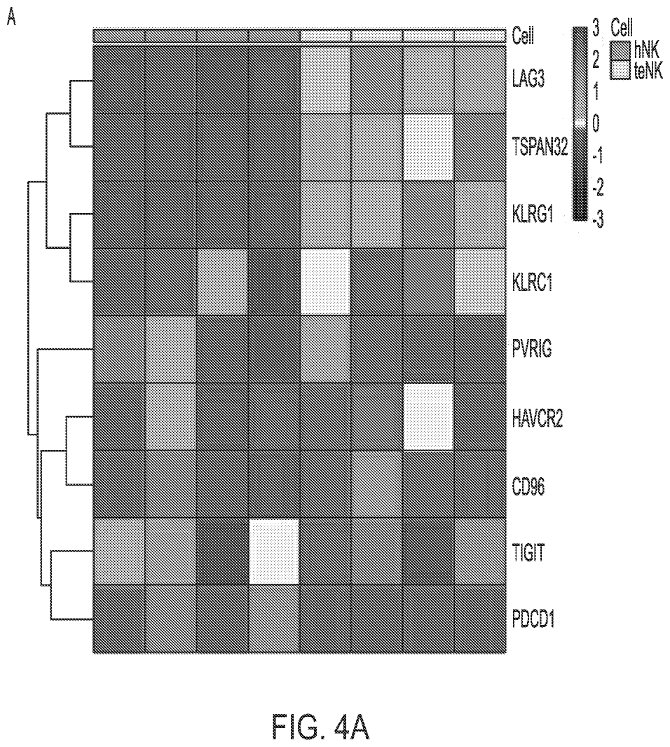

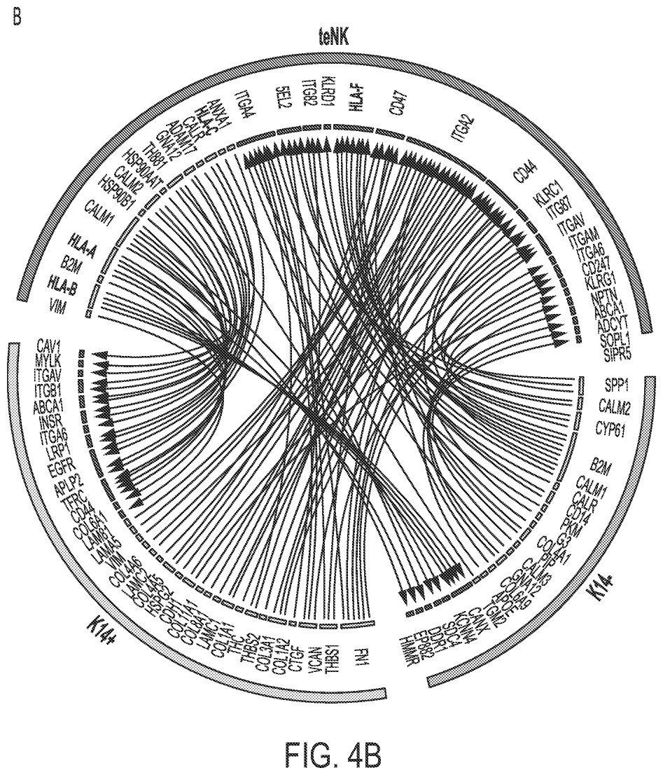

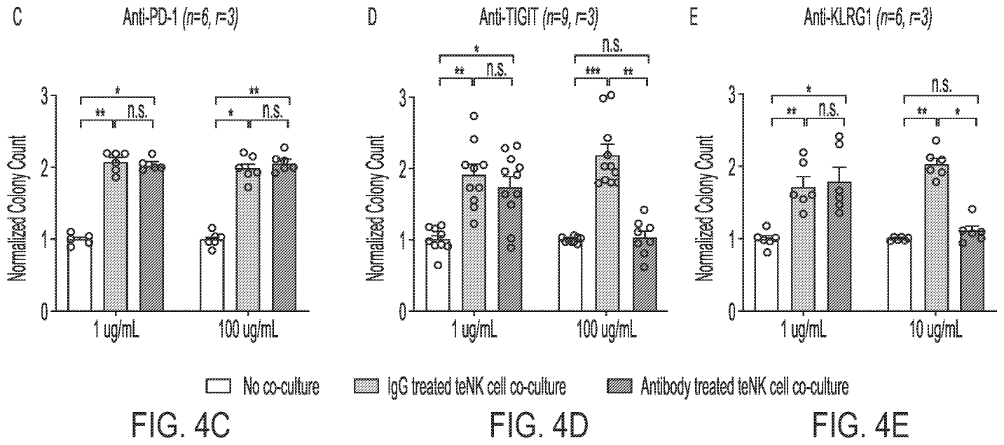

Next, experiments were carried out seeking to identify molecular strategies to reverse the metastasis promoting effect of teNK cells. Analysis of RNA-seq data revealed increased expression of inactivating receptors in teNK cells when compared with hNK cells (FIG. 4A). ligand-receptor pairs potentially responsible for signaling between NK cells and tumor cells were computationally identified. Applying the R package iTalk (Wang Y, et al. iTALK: an R Package to Characterize and Illustrate Intercellular Communication. bioRxiv. 2019:507871), which includes 2,648 known receptor-ligand pairs, ligand-receptor pairings between highly transcribed genes on K14+ and K14− cells and the most significantly expressed genes on teNK cells or hNK cells were found. Overall there were slightly more pairings between both types of NK cells and K14+ cells than with K14− cells (FIGS. 10C-D). Given the prior result showing that K14+ cells increase IFNg production in hNK cells, ligand-receptor interactions were identified that could lead to IFNg production on NK cells and apoptosis in K14+ cells. Ligand-receptor pairings that fit these criteria were TRAILR2-TRAIL, Caveolin-1-HRAS, and TRAF2-TNFSF4 on K14+ cells and hNK cells, respectively (FIG. 10E). Among ligand-receptor pairings on tumor cells and teNK cells, Cdh1 on K14+ cells were suggested to bind to Klrg1 on teNK cells (FIG. 4B).

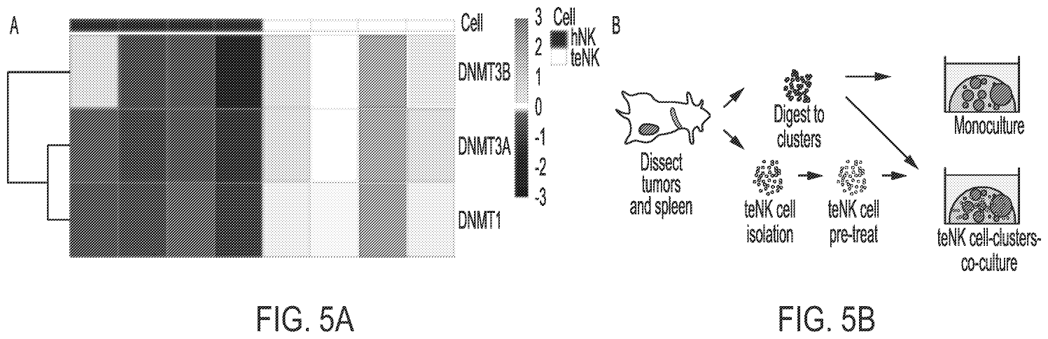

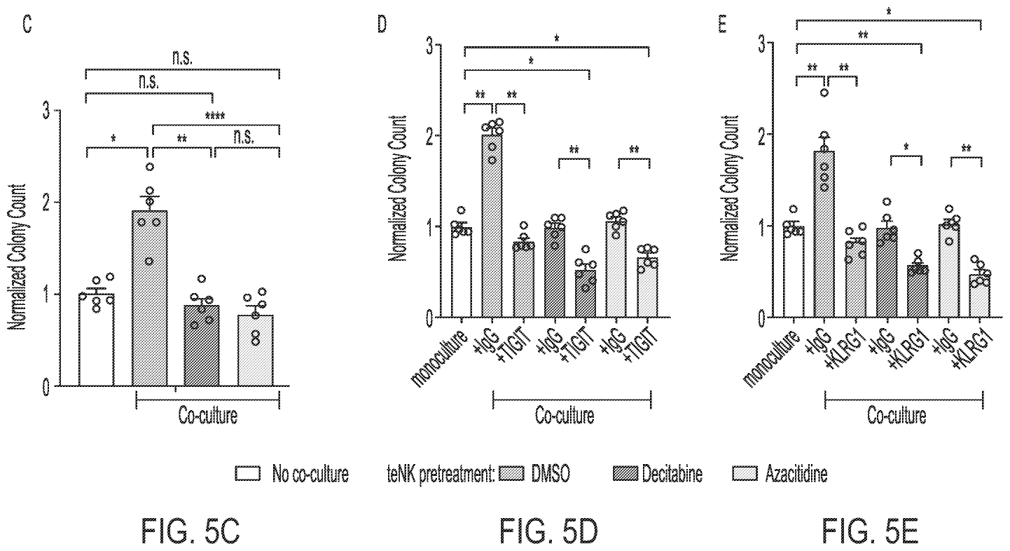

The capacity of candidate immunotherapies to reverse the effect of teNK cells on colony formation was investigated. Anti-PD-1 therapy has been reported to restore the function of anergic NK cells (Hsu J, et al. J Clin Invest. 2018; 128(10):4654-68). However, consistent with their limited clinical efficacy in breast cancer (Dirix L Y, et al. Breast Cancer Res Treat. 2018; 167(3):671-86), treatment with anti-PD-1 antibodies did not limit the colony promoting effect of teNK cells (FIG. 4C). Targeted receptors were next identified in the RNA expression analysis, specifically TIGIT and KLRG1. TIGIT is an emerging immune checkpoint on NK cells and T cells and anti-TIGIT therapies are in clinical trials (Zhang Q, et al. Blockade of the checkpoint receptor TIGIT prevents NK cell exhaustion and elicits potent anti-tumor immunity. Nat Immunol. 2018; 19(7):723-32). KLRG1, a marker of senescent NK cells and T cells and a recently reported inhibitor of NK cell function (Muller-Durovic B, et al. J Immunol. 2016; 197(7):2891-9), was highly expressed by teNK cells. Strikingly, treatment with either anti-TIGIT or anti-KLRG1 antibodies neutralized the effect of teNK cells and reduced colony formation (FIG. 4D-E). The broad gene expression differences between hNK and teNK cells provided the motivation to identify epigenetic regulators of NK cell state. It was found that DNA methyltransferases (DNMT1, DNMT3a, DNMT3b) were highly differentially expressed by teNK cells, relative to NK cells (FIG. 5A). Experiments were then carried out seeking to validate the functional significance of these findings in the co-culture models. To avoid potential tumor intrinsic impact of DNMT inhibition, teNK cells were pretreated with FDA approved DNMT inhibitors, decitabine and azacitidine, for 24 hours before co-culture (FIG. 5B). Pretreatment with DNMT inhibitors also neutralized the effect of teNK cells in co-culture (FIG. 5C) and reduced gene expression of TIGIT and KLRG1 (FIG. 10F). There is growing evidence of synergy between epigenetic modifiers and immune checkpoint inhibitors (Topper M J, et al. Nat Rev Clin Oncol. 2019). It was assessed whether the combination of FDA approved DNA methyltransferase inhibitors and anti-TIGIT or anti-KLRG1 would prevent colonies from being formed in co-culture. After pre-treatment with decitabine and azacytidine, treatment of co-cultures with anti-TIGIT (FIG. 5D) or anti-KLRG1 (FIG. 5E) blocking antibodies reduced the number of colonies below the monoculture controls.

Described herein are the results from studies seeking to understand the cellular and molecular basis of NK cell—cancer cell interactions during breast cancer metastasis. Clusters of K14+ cancer cells pioneer collective invasion and distant metastasis across breast cancer subtypes (Cheung K J, et al. Cell. 2013; 155(7):1639-51; Cheung K J, and Ewald A J. Science. 2016; 352(6282):167-9; and Cheung K J, et al. Proc Natl Acad Sci USA. 2016; 113(7):E854-63) and that K14 expression correlates with quantitative measures of invasion in human breast tumor samples (Padmanaban V, et al. PLoS Comput Biol. 2020; 16(1):e1007464). Described herein are results showing that NK cells are the most abundant early responder to cancer cell clusters in the lung, that K14+ cancer cells typically lack MHC Class I expression, and that hNK cells preferentially target K14+ cancer cells for cytotoxic death. The varied 3D culture assays revealed that hNK co-culture induced a mild reduction in tumor organoid growth, moderate reduction in organoid invasion, and a strong reduction in colony formation in culture and metastatic outgrowth in vivo. The effect on colony formation was increased further by treatment with antibodies targeting cell surface receptors on K14+ cells, presumably through ADCC. The relative impact of NK cells on cancer progression was most pronounced in assays modeling later stages of metastasis, similar to those seen in the adjuvant breast cancer setting. These observations support the view of NK cells as negative regulators of metastasis (Lopez-Soto A, et al. Cancer Cell. 2017; 32(2):135-54; and Krasnova Y, et al. Clin Immunol. 2017; 177(50-9)).

Until now, it remained unclear how metastases ever emerged from NK surveillance to reach clinical significance. The results show that after an initial period of complete control, the cancer cells eventually began to grow and invade. This observation led to the finding that teNK cells isolated from the spleens of tumor bearing mice were able to promote colony formation in vitro and metastasis formation in vivo. It is also shown that tiNK cells isolated from the primary tumor similarly promoted colony formation in vitro and that the NK education process could be replicated in culture with either murine or human NK cells. These results demonstrate that cancer cells can directly reprogram NK cells to promote metastatic colony formation adding to the concept of NK cell plasticity (Gao Y, et al. Nat Immunol. 2017; 18(9):1004-15) and to prior mechanisms of evasion of NK cell surveillance (Cherfils-Vicini J, et al. EMBO J. 2019; 38(11)). Just as other immune cells demonstrate a range of molecular and functional phenotypes that are guided by specific environmental contexts, the data described herein have shown that exposure to metastatic seeds is sufficient to alter the transcriptomic and functional state of NK cells.

Bulk RNA-seq comparisons of the transcriptomes of hNK and teNK cells were conducted. Upregulation of inhibitory receptors and a shift towards a resting NK state in teNK cells was observed. Next, the results show that antibodies directed towards either TIGIT or KLRG1 were sufficient to eliminate the metastasis-promoting effects of teNK cells and that the combination of DNMT inhibition and anti-TIGIT or anti-KLRG1 antibody treatment further reduced metastatic potential. These data demonstrate that cancer cells can convert NK cells to an alternative metastasis-promoting cell state and the role of KLRG1 in NK cell cooption. Further, the synergistic effects of epigenetic modification with inhibitory receptor blockade suggests a viable clinical strategy to activate tumor-exposed NK cells to target and eliminate breast cancer metastases.

The ex vivo models combine primary metastatic breast cancer cells with primary NK cells to allow the cellular and molecular dynamics of immune control and immune escape to be captured. These models can be readily adaptable to other cancer types and offer a scalable way to identify new molecular targets for NK cell-directed immunotherapies, test combination therapies, and inform their potential clinical utilization. Furthermore, the analysis of receptor-ligand pairing between teNK cells and tumor cells suggest a diverse range of additional drug targets for further validation. These results suggest that antibodies targeting NK inhibitory receptors could be effective in eliminating metastatic breast cancer cells, either alone or in combination with epigenetic therapies. Combined with the observation that NK cells are abundant early responders to disseminated breast cancer cells, the data provide preclinical rationale for the concept of NK cell directed immunotherapies in the adjuvant setting for breast cancer patients with high risk of metastatic recurrence.

Methods. Mouse lines and breeding. Study mice were female and were backcrossed and maintained on the FVB/n background. FVB/N-Tg(MMTV-PyVT)634Mul/J (MMTV-PyMT) (Guy C T, et al. Mol Cell Biol. 1992; 12(3):954-61), FVB-Tg(C3-1-TAg)cJeg/JegJ (C3(1)-Tag) (Maroulakou I G, et al. Proc Natl Acad Sci USA. 1994; 91(23):11236-40), B6.129(Cg)-Gt(ROSA)26Sortm4(ACTB-tdTomato, −EGFP)Luo/J (ROSAmT/mG), and NOD.Cg-Prkdcscid Il2rgtm1Wj1/SzJ (NSG) mice were obtained from the Jackson Laboratories. K14-actin-GFP mice were also used. For sorting of K14+ cells, MMTV-PyMT mice were crossed with K14-GFP-actin mice.

Tumor organoid and tumor cluster isolation. Primary tumor organoids were isolated from murine mammary tumors by step-wise mechanical disruption, enzymatic digestion, and differential centrifugation (Nguyen-Ngoc K V, et al. Methods Mol Biol. 2015; 1189(135-62)). Briefly, tumors were harvested from MMTV-PyMT or C3(1)-Tag mice with 20 mm tumors, mechanically disrupted with a scalpel, and digested on a shaker for 1 h at 37° C. in collagenase solution: (DMEM-F12 (10565-018; Gibco Life Technologies) with 2 mg/mL collagenase (C2139; Sigma-Aldrich), 2 mg/mL trypsin (27250-018; Gibco Life Technologies), 5% (vol/vol) FBS (F0926; Sigma-Aldrich), 5 μg/mL insulin (19278; Sigma-Aldrich), and 50 μg/mL gentamicin (15750; Gibco Life Technologies). The suspension was centrifuged at 400×g to remove cellular debris and undigested tissue, and the pellet was treated with 2 U/μL DNase (D4263; Sigma-Aldrich). The epithelial organoids were enriched and separated from stromal cells by a series of differential centrifugations, following which organoids of about 100-500 epithelial cells were obtained. Tumor cell clusters of 2-5 cells were obtained by taking organoids and further digesting them with 1×TrypLE (#12604013; Thermo Fisher) for 10 minutes at 37° C. Cells were resuspended in PBS (—Ca2+, —Mg2+), filtered through a 40 μm filter and resuspended again at a density of 2×106 cells/mL.

Natural killer cell isolation. Primary splenocytes were isolated by mechanical dissociation from spleens of wild-type FVB/n, MMTV-PyMT, or C3(1)-Tag mice, or from the tumors of MMTV-PyMT mice. NK cells isolated from WT FVB/n mice are defined as healthy NK (hNK) cells and those isolated from tumor bearing mice are defined as tumor-exposed NK (teNK) cells. NK cells isolated from MMTV-PyMT tumors are defined as tumor-infiltrating NK cells. The NK cells were purified from isolated splenocytes using EasySep Mouse NK Cell Isolation Kit per the manufacturer's protocol (#19855; Stemcell Technologies). The isolated cells were then positively selected by FACS by gating on CD49b+/CD3− cells and CD45+/CD49b+/CD3− for NK cells isolated from tumors. NK cells were cultured in media at 37° C. containing 200 U/ml recombinant mouse IL-2 (#402-ML-100, R&D Systems), 5 ng/ml recombinant mouse IL-15 (#447-ML-010, R&D Systems) or 5 ng/ml recombinant mouse IL-I5:IL-15R complex (#14-8152-62, Thermo Fisher) for 16 h before use in assays. Pretreatment of tumor-exposed NK cells were performed with decitabine and azacitidine at 1 μM doses for 24 hours (NCI Development Therapeutics Program, dtp.cancer.gov).

NK cell-organoid and NK cell-tumor cluster co-culture. Organoids were embedded at a density of 1.5 organoids/μL with 30:1 or 10:1 hNK or teNK cells (NK cells:tumor cells) into neutralized, fibrillar rat-tail collagen I (#354236, Corning) onto 24-well or 96-well glass bottomed plates over a 37° C. heating block to generate a NK cell-organoid co-culture. 30:1 or 10:1 NK cells:tumor cells. Tumor clusters were embedded at a density of 100 clusters/μL into Matrigel (#354230, BD Biosciences) onto 24-well or 96-well glass bottomed plates over a 37° C. heating block to generate NK cell-tumor cluster co-culture. Human NK cell-tumor cluster co-cultures were generated by embedding human NK-92 cells (ATCC CRL-2408) and MCF7 tumor cell clusters (ATCC HTB-22) into Matrigel. Gels were allowed to polymerize at 37° C. for an hour, after which media containing RPMI-1690 (#11875093, Gibco), 1% insulin-transferrin-selenium (#51500-056, Gibco), 1% penicillin-streptomycin (#P4333, Sigma), 2.4 nM FGF2 (#F0291, Sigma) and supplemented with 200 U/ml recombinant mouse IL-2, recombinant mouse 5 ng/ml IL-15, or 5 ng/ml recombinant mouse IL-I5:IL-15Ra complexes, was added to the wells. Culture-educated NK (ceNK) cells were produced by first generating a mTomato+hNK cell-tumor cluster co-culture for 48 hours. Culture-educated human NK (ceHuNK) cells were generated by first generating RFP+ human NK-92 by incubating cells with CellTracker Red (C34552, ThermoFisher) for 24 hours before co-culture. Co-culture gels were then digested by pipetting in cold PBS-EDTA buffer, comprised of PBS and 20 mM ethylenediaminetetraacetic acid (EDTA). The solubilized matrix-NK cell mix was centrifuged for 5 min at 400× g, 4° C., washed with PBS, and NK cells were isolated by FACS.

ADCC assay. ADCC assays were performed using tumor clusters isolated from MMTV-PyMT mice and co-cultures were formed with hNK cells at an intermediate 10:1 NK cell:tumor cell ratio. Prior to culture, tumor clusters were subsequently incubated with 5 μg/mL of CD44 antibody (#14-0441-82, eBioscience) or with media alone for another 30 minutes at 37° C., and washed twice with media to remove unbound antibodies. Colony formation was assessed at the end of 24 hours.

Differential Interference Contrast (DIC). DIC imaging was performed using a LD Plan-Neofluar 20×/0.4 Korr Ph2 objective lens, a Zeiss AxioObserver Z1, and an AxioCam MRM camera. DIC time-lapse movies were collected at 15-minute acquisition intervals for 24 hours, maintaining temperature at 37° C. and CO2 at 5%. Movie 1 shows live confocal imaging of hNK cells activating caspase in invading tumor cells. Movie 2 shows a differential interference contrast (DIC) timelapse movie of a MMTV-PyMT organoid in mono-culture. Movie 3 shows a DIC timelapse movie of a MMTV-PyMT organoid in co-culture with hNK cells. Movie 4 shows a DIC timelapse movie of a C3(1)-Tag organoid in mono-culture. Movie 5 shows a DIC timelapse movie of a C3(1)-Tag organoid in co-culture with hNK cells.

Quantification of organoid invasion, organoid growth, and colony formation. Invasion of organoids was quantified by manually tracing organoid boundaries from DIC images and measuring its circularity with ImageJ. Circularity is defined as the ratio of the square of the perimeter to 4π times the area. A circle, which has the maximum area for a given perimeter, has a value of 1; organoids with multiple invasive strands have much higher values. Inverse circularity is graphed to provide a relationship between increased invasion and increased inverse circularity (FIG. 1D, FIG. 1E). For growth, paired images for each organoid were obtained at 0 hours and 24 hours post-plating and growth is represented as a fold change in projected area in 24 hours. Colony formation was performed by counting the tumor colonies in each well across multiple Z planes. FIG. 1G is a representation of colonies counted.

K14+-tumor cells/NK co-culture assay. Single cell suspensions of K14+ and K14− cells were generated from MMTV-PyMT; K14-actin-GFP mammary tumor organoids and separated by FACS. Equal numbers of K14+ cells or K14− cells were placed in suspension with hNK cells isolated from ROSAmTmG mice at a 10:1 tumor cell:NK cell ratio for 4 hours at 37° C. Samples were fixed and permeabilized using BD CytoFix/CytoPerm (#554714, BD Biosciences) and analyzed by flow cytometry for IFNγ.

Flow cytometry and cell sorting. MMTV-PyMT; K14− actin-GFP organoids were harvested for tumor and subsequently dissociated into single cell suspensions using TrypLE. The GFP+/K14+ cells were analyzed and sorted using either a MoFlo Legacy or XDP Cytometer (Beckman Coulter, Miami, FL, USA). Data acquisition and sort were performed by Summit software. hNK cells were isolated as CD3−/CD49b+/mTomato+ cell population from the spleens of ROSAmTmG mice. Propidium iodide (PI) or DAPI was used as a viability marker for the sorts. FCS files generated from FACS experiments were re-analyzed by FlowJo software for data representation.

Tail vein assays. Mammary tumor organoids from MMTV-PyMT; ROSAmTmG were trypsinized into small clusters using TrypLE (Thermo Fisher) at 37° C. for 10 minutes. Cells were resuspended in DMEM-F12 at a concentration of 106 cells/mL. Host FVB/n and NSG mice for these experiments were 6-10 week old female mice. The tail veins of these mice were dilated by exposure to a heat lamp for ˜1 minute. Using a 26.5-gauge needle, 200 μL of cells were injected via the tail vein of the mouse. In NSG mice, tail vein assays were followed by adoptive transfer of 500,000 NK cells in 200 μL of PBS on the next day. Cytokine support with 1 μg recombinant mouse IL-15:IL-15Ra complexes ((#14-8152-62, Thermo Fisher) was delivered intraperitoneally for three days. Lungs from mice were collected 7 days from the date of injection and examined for macrometastases. Macrometastases were counted based on expression of mTomato+ under the dissection microscope. Representative images in FIG. 3C′ were collected using an iPhone X.

Immunofluorescence. Organoid co-cultures and tumor clusters cultured in 3D collagen I were fixed using 4% paraformaldehyde (PFA; Electron Microscopy Sciences, 15714S), while colonies in Matrigel were fixed using 1% PFA for 10 mins. Residual formaldehyde was washed two times in D-PBS. The gels were permeabilized using 0.5% TritonX-100 (X100-500ML, Sigma) for 30 minutes at room temperature. Samples were blocked for 2 hours with 10% FBS/1% BSA/0.2% TritonX/PBS at room temperature, incubated with primary antibodies diluted in 1% FBS/1% BSA/0.2% TritonX/PBS overnight at 4° C., then washed three times for 10 mins each using PBS. Secondary antibodies were added diluted in 1% FBS/1% BSA/0.2% TritonX/PBS, and incubated for 3 hours at room temperature. Samples were washed three times with PBS and stored at 4° C. until imaged. Confocal imaging was conducted on a spinning disk microscope (Solamere Technology Group Inc.) with a Prime 95B Scientific CMOS camera (Photometrics). A LD Plan-Neofluar 20×/0.4 Korr Ph2 objective lens (Zeiss) was used for single and time-lapse image acquisition. For time-lapse imaging, images were acquired at 20-min time intervals with 15-20. For z stacks, 2-μm spacing was used. Acquisition of both time-lapse and still images was performed using μManager (Edelstein A, et al. Curr Protoc Mol Biol. 2010; Chapter 14(Unit14 20)).

For confocal movies, hNK cells were isolated from ROSAmTmG mice, the tumor cell nuclei are labeled using Sir-DNA (#CY-SC007, Cytoskeleton) and caspase activity was assessed using CellEvent Caspase-3/7 Green Detection Reagent (#C10423, Thermo Fisher). Images were collected every 10 minutes using 2-μm spacing for 24 hours under standard incubation conditions. Videos were collected in parallel using one to three channels (excitation at 488 nm, 561 nm, and 647 nm). Imaris 8 (Bitplane) was used to analyze videos, export individual TIFFs, and adjust brightness and contrast of the images in each channel to maximize the clarity. ImageJ and Imaris were used to adjust brightness and contrast of the images in each channel to maximize clarity, place scale bars, and export as TIFFs. Image adjustments were always made across entire images.

Prior to lung harvest, cardiac perfusion with PBS and 1% PFA was performed. Lungs from tail vein assays were then harvested and fixed in 1% PFA for 4 hours at 4° C. Fixed tumor cells and lungs were transferred into 25% sucrose/PBS overnight at 4° C., embedded into Optimal Cutting Temperature compound (#4583, TissueTek) and frozen at −80° C. Sections (20 μm thickness) were cut onto Superfrost Plus Gold Microscope slides (Thermo Fisher Scientific, 15-188-48) at −20° C. using a cryostat. The OCT was removed from these slides by incubating with PBS for 1 hour at room temperature. The sections were processed and stained identical to gel embedded co-cultures, covered with #1.5 High Precision Cover Glasses (#CG15KH, Thor Labs), and images were acquired using a Zeiss Axio Scan.Z1 and analyzed with the Zen Imaging software.

Antibodies. Primary antibodies used in the studies include anti-NK1.1 (1:200; #553162, BD Biosciences), anti-F4/80 (1:200; #123122, Biolegend), anti-neutrophil-elastase (1:200; #ab68672, Abcam), anti-keratin-14 (1:200; #PRB-155P, Covance), anti-CD49b (1:200; StemCell Technologies), anti-CD3 (1:200; StemCell Technologies), anti-CD44 (1:200; #14-0441-82, eBioscience), anti-IFN-gamma (1:100; #11-7311-82, Thermo Fisher), and DAPI (1:1000; Invitrogen, D571). Secondary antibodies used were AlexaFluor-conjugates (1:200; Invitrogen). Treatment of mono-cultures and co-cultures described in the main text were performed with anti-TIGIT antibody (clone 1G9, #BE0274, BioXCell) and anti-KLRG1 antibody (clone 2F1, #16-5893-82, Thermo Fisher).

RNA Extraction and Quantitative PCR. RNA was extracted with the RNeasy Mini Kit (Qiagen #74104) following the manufacturer's protocol. cDNA was synthesized from 100 ng total RNA using the SuperScript IV VILO Master Mix (Thermo Fisher #11766050). Synthesized cDNA was diluted in RNAase-free water prior to RT-qPCR. Quantitative PCR was conducted using the SsoAdvanced Universal SYBR Green Supermix (BioRad #1725271) with 500 pg cDNA and 500 nM primers per reaction. Reactions were run in triplicate on a CFX96 Touch Real-Time PCR Detection System (BioRad). Target gene expression values were normalized to GAPDH expression and fold change was calculated as 2-ΔΔCt. RT-qPCR primers used were: KLRG1-Forward, 5′-GCTCACATCTCCTTACATTTCCG-3′ (SEQ ID NO: 1), KLRG1-Reverse, 5′-TCCTCAAGCCGATCCAGTA-3′ (SEQ ID NO: 2), TIGIT-Forward, 5′-CTGCCTTCCTCGCTACAG-3′ (SEQ ID NO: 3), TIGIT-Reverse, 5′-GTAAGATGACAGAGCCACCTTC-3′ (SEQ ID NO: 4).

High throughput RNA-sequencing and gene-set analysis. For each replicate, primary hNK cells and teNK cells were isolated and cultured in cytokine supplemented media for 12-16 hours. The cells were then snap-frozen and total RNA was extracted using RNeasy (Qiagen, 74104). The sequencing library was prepared using the TruSeq Stranded mRNA Sample Kit (Illumina, RS-122-2101). Briefly, at least 400 pg of total RNA isolated per sample was converted to double-stranded cDNA, end-repaired, A-tailed, and ligated with Illumina indexed adapters. The PCR amplified library was purified using Agencourt RNAClean XP (Beckman Coulter, A63987) magnetic beads and run out on Agilent High Sensitivity DNA Chip for quality check. The library was then sequenced in a paired-end 150 bp cycle using Illumina NextSeq 500 (The Johns Hopkins School of Medicine Deep Sequencing and Microarray Core Facility).

The mouse genome was obtained in FASTA format (GRCm38) from Ensembl version 95 and gene set annotation in GTF format. The hisat2 indices were built from the genome index using hisat2-build (Kim D, et al. Nat Methods. 2015; 12(4):357-60) from Hisat2 version 2.1.0. Raw RNAseq paired-end reads were aligned to the genome using hisat2 and trimming the first base (using optional flag −5 1). The total reads per sample ranged from 37-62 million and the alignment mapping rate was 88-90%. The DNA fragments had been labeled with Unique Molecular Identifiers (UMIs). NuDup (version 2.3) was used to mark the UMIs and keep the read pair with the highest mapping quality. HTSeq was used to count reads mapping to individual genes by processing the sorted bam files with accepted reads (Anders S, et al. Bioinformatics. 2015; 31(2):166-9).

DESeq2 (Love M I, et al. Genome Biol. 2014; 15(12):550) was used to estimate differential gene expression between hNK and teNK cells from the counts generated by HTSeq. Standard DESeq2 parameters were used to exclude genes with no reads and those with p-values set to the nominal value of 1. Additionally, the genes with 10 or fewer total reads were removed.

External databases. Gene Ontology. Gene ontology (GO) pathway analysis was carried out using the R package goseq (Young M D, et al. Genome Biol. 2010; 11(2):R14).

ImmuCC and CIBERSORT. Mouse specific immune cell signatures from ImmuCC (15) were downloaded, based on the expression of 511 genes, and were loaded along with the list of genes differentially expressed between teNK and hNK into CIBERSORT (Newman A M, et al. Nat Methods. 2015; 12(5):453-7). CIBERSORT uses signature expression profiles for cells to deconvolute the gene expression from a mixture of cells. The algorithm quantifies relative levels of distinct cell types.

iTALK software package. iTALK (Wang Y, et al. iTALK: an R Package to Characterize and Illustrate Intercellular Communication. bioRxiv. 2019:507871) is an R package that is based on 2,648 unique receptor-ligand pairs. Using the previously published RNA-seq data for K14+ and K14− cells (Cheung K J, et al. Proc Natl Acad Sci USA. 2016; 113(7):E854-63) with mean expression greater than 10 and the differentially expressed genes identified in this study the algorithm identified 648 receptor-ligand pairs between hNK and K14+ and K14− cells and 853 pairs between teNK and K14+ and K14− cells. The receptor-ligand pairs were ordered based on the product of the mean expression for each cell type.

Statistical analysis. Statistical tests were performed on GraphPad by Prism. The tests used are described in figure legends. p-value less than 0.05 was considered significant.

### Example 2: NK Cell Isolation, Activation, and Expansion, Usually Prepared One Day Before Co-Culture with Tumor Cells

Isolation and activation of mouse splenic natural killer (NK) cells from mice that are wild-type or with mammary tumors. Use mice between 8-12 weeks old.

Materials for use: One mouse, One sterilized standard forceps, One sterilized Iris scissors, Polystyrene petri dish, 5 mL syringe plunger, 10 mL syringe, 50 mL Eppendorf conical tube, Ice bucket, 70 μm cell filter, RMPI 1640 Media (61/870,036, ThermoFisher), EasySep Mouse NK Cell Isolation Kit (19855; StemCell Technologies), and EasySep Magnet (18000; StemCell Technologies).

NK cell mouse media containing: RPMI 1640 media above, 10% (vol/vol) heat inactivated fetal bovine serum, 1% (vol/vol) 100× solution penicillin/streptomycin, 10 mM HEPES buffer, 200 U/mL recombinant mouse IL-2 (402-ML-100; R&D Systems), and 5 ng/mL recombinant mouse IL-15:IL-15R complex (14-8152-62; Thermo Fisher Scientific)

Procedure:
1) Euthanize the mouse in a CO2-saturated chamber for 3-5 min followed by cervical dislocation.
2) Pin the mouse face up to a protected Styrofoam board.
3) Wet the mouse thoroughly with 70% EtOH.
4) Use the Spencer Ligature scissors to cut along the ventral midline from the groin to the diaphragm. Puncture the peritoneum without puncturing any organs.
5) Use the standard forceps to pull back the skin and peritoneal folds. Grasp the spleen and dissect whole spleen out.
6) Using a 5 mL syringe plunger, mechanically dissociate the whole spleen into 10 cc of RPMI 1640 media.
7) Using a 10 mL syringe, filter and transfer isolate to a 50 mL Eppendorf conical tube. Keep tube on ice.
8) Using an EasySep Mouse NK Cell Isolation Kit, isolate NK cells per manufacturer's protocol using EasySep Magnet.
9) Transfer freshly isolated NK cells to 10 mL of NK cell mouse media.
10) Activate freshly isolated mouse NK cells by incubating for 16 hours in a cell culture incubator maintained at 5% CO₂ and 37° C.

Isolation and Activation of Natural Killer (NK) Cells from Mouse Mammary Tumors.

Materials for use: One mouse with mammary tumors, One standard forceps, One Spencer Ligature scissors, One Iris scissors, Sterile scalpel, Polystyrene petri dish, Benchtop incubator orbital shaker, Benchtop swinging bucket centrifuge, Cell culture incubator, Ice bucket, 15 mL Eppendorf conical tube, 50 mL Eppendorf conical tube, and DMEM/F12 media (Life Technologies #10565-018); Collagenase solution per mouse, filter sterilized through 0.2 μm filter, containing: 9 mL DMEM/F12, 5004, FBS, 54, insulin (10 mg/mL stock), 104, gentamycin (50 mg/mL stock), 2004, collagenase (100 mg/mL stock), and 2004, trypsin (100 mg/mL stock); BSA solution, filter sterilized, containing: 46 mL DPBS, and 4.1 mL BSA; 2000 U/mL DNase dissolved in PBS; Organoid media containing: DMEM/F12, 5 mL penicillin/streptomycin, 5 mL ITS, and 2.5 nM FGF2; RBC Lysis Buffer (#420301, Biolegend); Phosphate buffered saline (10010023; Thermo Fisher Scientific); EasySep Mouse NK Cell Isolation Kit (19855; StemCell Technologies); EasySep Magnet (18000; StemCell Technologies); Anti-CD49b antibody (clone DX5, StemCell Technologies); Anti-CD3e antibody (clone 145-2C11, StemCell Technologies); Flow sorter; Antibody dilution buffer containing: Phosphate buffered saline (10010023; Thermo Fisher Scientific) and 1% (vol/vol) fetal bovine solution; NK cell mouse media containing: RPMI 1640 media above, 10% (vol/vol) heat inactivated fetal bovine serum, 1% (vol/vol) 100× solution penicillin/streptomycin, 10 mM HEPES buffer, 200 U/mL recombinant mouse IL-2 (402-ML-100; R&D Systems), and 5 ng/mL recombinant mouse IL-15:IL-15R complex (14-8152-62; Thermo Fisher Scientific).

Procedure:
1) Sterilize dissecting area with 70% ethanol.
2) Sterilize dissecting tools by heat in a glass bead sterilizer.
3) Euthanize the mouse in a CO2-saturated chamber for 3-5 min followed by cervical dislocation.
4) Pin the mouse face up to a protected Styrofoam board.
5) Wet the mouse thoroughly with 70% EtOH.
6) Use Spencer Ligature scissors to cut along the ventral midline from the groin to the chin. Cut only the skin and do not puncture the peritoneal cavity.
7) Make four incisions from the midline cut towards the four legs.
8) Use standard forceps to pull back the skin one side at a time to expose mammary tumors. Use dorsal side of forceps to separate skin from the peritoneum.
9) Use the standard forceps and Iris scissors to grasp and pull out mammary tumors from both right and left sides. Pool tumors in a sterile Petri dish.
10) In a sterile hood, mince mammary tumors with a scalpel 50 times per mouse.
11) Use the scalpel to transfer the minced tumors to the collagenase solution in a 50 mL tube.
12) Shake the suspension at 180 rpm for 1 hour at 37° C. on the incubator orbital shaker.
13) Spin the 50 mL Eppendorf conical tube 10 minutes at 1500 RPM in the centrifuge at room temperature.
14) Aspirate supernatant from the tube.
15) Add 8 mL of DMEM/F12 and 80 uL of the DNase solution.
16) Invert conical tube for 1 minute to mix.
17) Add in 12 mL of DMEM/F12 and gently pipet up and down 5 times.
18) Spin down conical for 10 minutes at 1500 RPM in the centrifuge at room temperature.
19) Aspirate supernatant from the tube.
20) Add 10 mL DMEM/F12 to the remaining tissue suspension.
21) Precoat a 15 mL Eppendorf conical tube with the BSA solution. Remove BSA solution.
22) Transfer suspension from step #20 to precoated 15 mL Eppendorf conical tube.
23) Centrifuge 15 mL Eppendorf conical tube for 1500 RPM for 3 seconds at room temperature.
24) Collect supernatant in new 50 mL Eppendorf conical tube. Centrifuge for at 350 g for 10 minutes at room temperature.
25) Aspirate supernatant and treat remaining suspension with 5 mL 1×RBC lysis buffer for 5 minutes at room temperature.
26) Stop reaction by diluting RBC lysis buffer with 30 mL of phosphate buffered saline.
27) Centrifuge suspension at 350 g and discard supernatant.
28) Resuspend pellet to a cell density of 1×10⁸ cells/mL.
29) Using an EasySep Human NK Cell Isolation Kit, isolate NK cells per manufacturer's protocol using EasySep magnet.
30) Incubate cells with CD49b and CD3 antibodies for 30 minutes at a 1:200 dilution with antibody dilution buffer.
31) Perform a wash with phosphate buffered saline and centrifuge at 350 g for 10 minutes. Repeat for three washes.
32) Perform fluorescent activated cell sorting using a flow cytometer cell sorter on the gated populations CD49b+CD3− cells.
33) Transfer freshly isolated NK cells to 10 mL of NK cell mouse media.
34) Activate freshly isolated mouse NK cells by incubating for 16 hours in a cell culture incubator maintained at 5% CO₂ and 37° C.

Isolation and Activation of Natural Killer (NK) Cells from Human Peripheral Blood Mononuclear Cells (PBMCs).

Materials for use: Frozen human PBMCs, 50 mL Eppendorf conical tube, Ice bucket, RMPI 1640 Media (#61870036, ThermoFisher), EasySep Human NK Cell Isolation Kit (#17955; StemCell Technologies), EasySep Magnet (#18000; StemCell Technologies), Anti-CD56 antibody (clone 56C04, ThermoFisher Scientific), Anti-CD3 antibody (clone OKT3, ThermoFisher Scientific); Lymphocyte buffer containing: Phosphate buffered saline (10010023; ThermoFisher Scientific), 2% (vol/vol) fetal bovine solution, and 1 mM EDTA; Antibody dilution buffer containing: Phosphate buffered saline (10010023; Thermo Fisher Scientific), and 1% (vol/vol) fetal bovine solution; NK cell primary human media containing: RMPI 1640 Media (61/870,036, ThermoFisher), 1% (vol/vol) 100× solution penicillin/streptomycin, 10 mM HEPES buffer, 0.1 mM 2-mercaptoethanol, 12.5% (vol/vol) horse serum, 12.5% (vol/vol) fetal bovine serum, 0.2 mM inositol, 500 U/mL recombinant mouse IL-2 (402-ML-100; R&D Systems), and 50 ng/mL recombinant human IL-15 (#PHC9154, ThermoFisher); and 25 ng/mL recombinant human IL-21(#200-21, Peprotech).

Procedure:
1) Thaw frozen human PBMCs in Lymphocyte Buffer for 20 minutes before isolation.
2) Prepare sample at 5×10⁷ cells/mL in 2 mL of Lymphocyte Buffer.
3) Using an EasySep Human NK Cell Isolation Kit, isolate NK cells per manufacturer's protocol using EasySep magnet.
4) Incubate cells with CD56 and CD3 antibodies for 30 minutes at a 1:200 dilution with antibody dilution buffer.
5) Perform a wash with phosphate buffered saline and centrifuge at 350 g for 10 minutes. Repeat for three washes.
6) Perform fluorescent activated cell sorting using a flow cytometer cell sorter on the gated populations CD49b+CD3− cells.
7) Transfer freshly isolated NK cells to 10 mL of NK cell human media.
8) Activate freshly isolated mouse NK cells by incubating for 10 days in a cell culture incubator maintained at 5% CO2 and 37° C. Leaves cells undisturbed until media exchange. Exchange media every 4 days by centrifuging to pellet at 350 g for 10 minutes. Aspirate supernatant and resuspend with fresh media.
9) On day 12, centrifuge cells down, add IL-21 to the media. Continuing culturing cells with IL-21 after day 12 until experimental use.

Culture and expansion of NK-92 human NK cell line. Materials required: NK-92 (CRL-2407, ATCC); Cell culture incubator; Corning T-25 flask (#431463, Corning); NK-92 cell media containing: Alpha Minimum Essential Media, 2 mM L-glutamine, 1.5 g/L sodium bicarbonate, 0.2 mM inositol, 0.1 mM 2-mercaptoethanol, 0.02 nM folic acid, 1% (vol/vol) 100× solution penicillin/streptomycin, 12.5% (vol/vol) horse serum, 12.5% (vol/vol) fetal bovine serum, and 100 U/mL recombinant mouse IL-2 (402-ML-100; R&D Systems).

Procedure:
1) Pre-warm (37° C.) and equilibrate NK-92 cell media to temperature before plating cells.
2) Move the cell suspension to a conical tube. Pipette gently to break up the clumps and form a single cell suspension.
3) Count cells. Cells should be maintained at a density of 0.5×10⁶ cells/mL.
4) Centrifuge at 300 g (1300 rpm) for 7 min to pellet the cells.
5) Resuspend cells in an appropriate amount of NK-92 cell media and move to a culture flask. Place in a cell culture incubator maintained at 5% CO₂ and 37° C.

### Example 3: Co-Culture of Activated NK Cells with Tumor Cells

Preparation of organoids from mouse mammary tumors. Materials for use: One mouse with mammary tumors, One standard forceps, One Spencer Ligature scissors, One Iris scissors, Sterile scalpel, Polystyrene petri dish, Benchtop incubator orbital shaker, Benchtop swinging bucket centrifuge, Cell culture incubator, Ice bucket, 15 mL Eppendorf conical tube, 50 mL Eppendorf conical tube, DMEM/F12 media (Life Technologies #10565-018); Collagenase solution per mouse, filter sterilized through 0.2 μm filter, containing: 9 mL DMEM/F12, 5004, FBS, insulin (10 mg/mL stock), 104, gentamycin (50 mg/mL stock), 2004, of 100 mg/mL collagenase (#C2139, Sigma) (#C2139, Sigma), and 2004, trypsin (100 mg/mL stock); BSA solution, filter sterilized, containing: 46 mL DPBS, and 4.1 mL BSA; 2000 U/mL DNase dissolved in PBS; Mouse organoid media containing: DMEM/F12, 5 mL penicillin/streptomycin, 5 mL ITS, and 2.5 nM FGF2 (#F0291, Sigma).

Procedure:
1) Sterilize dissecting area with 70% ethanol.
2) Sterilize dissecting tools by heat in a glass bead sterilizer.
3) Euthanize the mouse in a CO2-saturated chamber for 3-5 min followed by cervical dislocation.
4) Pin the mouse face up to a protected Styrofoam board.
5) Wet the mouse thoroughly with 70% EtOH.
6) Use Spencer Ligature scissors to cut along the ventral midline from the groin to the chin. Cut only the skin and do not puncture the peritoneal cavity.
7) Make four incisions from the midline cut towards the four legs.
8) Use standard forceps to pull back the skin one side at a time to expose mammary tumors. Use dorsal side of forceps to separate skin from the peritoneum.
9) Use the standard forceps and Iris scissors to grasp and pull out mammary tumors from both right and left sides. Pool tumors in a sterile Petri dish.
10) In a sterile hood, mince mammary tumors with a scalpel 50 times per pooled sample.
11) Use the scalpel to transfer the minced tumors to the collagenase solution in a 50 mL tube.
12) Shake the suspension at 180 rpm for 1 hour at 37° C. on the incubator orbital shaker.
13) Spin the 50 mL Eppendorf conical tube 10 minutes at 1500 RPM in the centrifuge at room temperature.
14) Aspirate supernatant from the tube.
15) Add 8 mL of DMEM/F12 and 80 uL of the DNase solution.
16) Invert conical tube for 1 minute to mix.
17) Add in 12 mL of DMEM/F12 and gently pipet up and down 5 times.
18) Spin down conical for 10 minutes at 1500 RPM in the centrifuge at room temperature.
19) Aspirate supernatant from the tube.
20) Add 10 mL DMEM/F12 to the remaining tissue suspension.
21) Precoat a 15 mL Eppendorf conical tube with the BSA solution. Remove BSA solution.
22) After allowing heavy particles to settle, collect supernatant from step #20 and transfer to precoated 15 mL Eppendorf conical tube.
23) Centrifuge 15 mL Eppendorf conical tube for 1500 RPM for 3 seconds at room temperature, removing the aspirate and resuspending in 10 mL DMEM/F12 each time for a total of 4 times.
24) Resuspend final purified mouse tumor organoid isolate in 10 mL mouse organoid media and place in a cell culture incubator maintained at 5% CO₂ and 37° C.

Preparation of organoids from human breast tumors. Materials for use: Human breast tumor, Sterile scalpel, Polystyrene petri dish, Benchtop incubator orbital shaker, Benchtop swinging bucket centrifuge, Cell culture incubator, Ice bucket, 15 mL Eppendorf conical tube, 50 mL Eppendorf conical tube, and DMEM/F12 media (Life Technologies #10565-018); Human collagenase solution per human breast tumor sample, filter sterilized through 0.2 μm filter, containing: 18 mL RPMI 1640 (#D6546, Sigma), 200 μL Pen-Strep (1:100 dilution) (Sigma P4333), 200 μL of 10 mM Hepes Buffer, 1 mL fetal bovine serum, and 400 μL of 100 mg/mL collagenase (#C2139, Sigma); PBSA Wash; 10 mL of 100× penicillin/streptomycin; 10 mL Fungizone (#15290-018, Life Technologies); 500 mL DPBS; BSA solution, filter sterilized, containing: 46 mL DPBS and 4.1 mL BSA; 2000 U/mL DNase dissolved in PBS; Isolated NK cells from mouse or human sources.

Organoids isolated from mouse mammary tumors or human patient-derived xenografts. Materials: Benchtop swinging bucket centrifuge; Cell culture incubator; DMEM/F12 media (Life Technologies #10565-018); lx TrypLE (#12604013; Thermo Fisher Scientific); Phosphate buffered saline (#10010023; Thermo Fisher Scientific); Human organoid medium: 100 ml DMEM with 4500 mg/L glucose, sodium pyruvate, and sodium bicarbonate, without L-glutamine, liquid, sterile-filtered, suitable for cell culture: (#D6546, Sigma), 1 mL of 100× GlutaMAX, 1% (vol/vol) 100× penicillin-streptomycin, 10 mM Hepes buffer, 0.075% (vol/vol) BSA, 10 ng/ml Cholera Toxin, 0.47 ug/ml Hydrocortisone, 5 ug/ml human insulin solution, and 5 ng/ml EGF.

Procedure:
1) In a sterile hood, mince human breast tumor sample with a scalpel 50 times per pooled sample.
2) Use the scalpel to transfer the minced tumors to the human collagenase solution in a 50 mL tube.
3) Shake the suspension at 200 rpm for 1 hour at 37° C. on the incubator orbital shaker.
4) Spin the 50 mL Eppendorf conical tube 10 minutes at 1500 RPM in the centrifuge at room temperature.
5) Aspirate supernatant from the tube.
6) Add 8 mL of DMEM/F12 and 80 uL of the DNase solution.
7) Invert conical tube for 1 minute to mix.
8) Add in 12 mL of DMEM/F12 and gently pipet up and down 5 times.
9) Spin down conical for 10 minutes at 1500 RPM in the centrifuge at room temperature.
10) Aspirate supernatant from the tube.
11) Add 10 mL DMEM/F12 to the remaining tissue suspension.
12) Precoat a 15 mL Eppendorf conical tube with the BSA solution. Remove BSA solution.
13) After allowing heavy particles to settle, collect the supernatant from step #11 and transfer to precoated 15 mL Eppendorf conical tube.
14) Centrifuge 15 mL Eppendorf conical tube for 1500 RPM for 4 seconds at room temperature, removing the aspirate and resuspending in 10 mL DMEM/F12 each time for a total of 4 times.
15) Resuspend final purified mouse tumor organoid isolate in 10 mL human organoid media and place in a cell culture incubator maintained at 5% CO2 and 37° C.

Preparation of tumor clusters from mouse mammary tumor organoids or primary human breast tumor organoids. Materials for use: Organoids isolated from mouse mammary tumors or human breast tumors; Benchtop swinging bucket centrifuge; Cell culture incubator; DMEM/F12 media (#10565-018, Life Technologies); lx TrypLE (#12604013; Thermo Fisher Scientific);

Phosphate buffered saline (10010023; Thermo Fisher Scientific); Mouse organoid media (disclosed herein); and Human organoid media (disclosed herein).

Procedure:
1) Using isolated organoids, digest further with 2 mL of 1×TrypLE in a 15 mL Eppendorf conical tube for 10 minutes at 37° C.
2) Stop TrypLE digestion by adding 6 mL of DMEM/F12 to tube. Centrifuge at 1500 RPM for 10 minutes.
3) Aspirate supernatant and resuspend in 10 mL organoid media and place in a cell culture incubator maintained at 5% CO₂ and 37° C.

Co-culture of mouse or human natural killer cells or NK-92 cell lines with mouse or human tumor organoids or cell clusters or human tumor cell lines in collagen or 3D Matrigel. Materials for use: Activated NK cells; Organoids isolated from mouse mammary tumors or human patient-derived xenografts; Benchtop swinging bucket centrifuge; Cell culture incubator; Microtube incubator with two blocks set to 37° C.; 24-well glass bottom plates; 96-well glass bottom plates; DMEM/F12 media (Life Technologies #10565-018); lx TrypLE (#12604013; Thermo Fisher Scientific); Phosphate buffered saline (#10010023; Thermo Fisher Scientific); 15 mL Eppendorf conical tube; Collagen Solution titrated with sodium bicarbonate or HCl to a pH of 7.4: 375 uL of 10×DMEM (#354236, Sigma), 100 uL of sodium bicarbonate, 3 mL of Rat tail Collagen I solution (#354236, Corning); Matrigel (#354230, BD Biosciences); Co-culture media for mouse NK cells and mouse mammary tumor cells: RPMI 1640 media above, 10% (vol/vol) heat inactivated fetal bovine serum, 1% (vol/vol) 100× solution penicillin/streptomycin, 10 mM HEPES buffer, 200 U/mL recombinant mouse IL-2 (402-ML-100; R&D Systems), 5 ng/mL recombinant mouse IL-15:IL-15R complex (14-8152-62; Thermo Fisher Scientific), and 2.5 nM FGF2 (#F0291, Sigma); Co-culture media for primary human NK cells and primary human tumor cells: A 50:50 mix of: NK cell primary human media, including 25 ng/mL recombinant human IL-21 and Human organoid media disclosed herein; and NK-92 cell media.

Procedure:
1) Prepare the collagen solution or thaw Matrigel.
2) If plating in collagen, make underlays by pipetting 104, of collagen into a well of a 24-well plate or 24, of collagen into a well of a 96-well plate 1 hour before plating co-cultures.
3) Co-culture at a density of 30 tumor cells to 1 NK cell. Add tumor and NK cells suspended in their appropriate volume into 15 mL Eppendorf conical tube.
4) Centrifuge cell mixture at 350 g for 10 minutes.
5) Aspirate supernatant and leave cell pellet.
6) Resuspend pellet with collagen or Matrigel. Plate 1004, gels in 24-well plates or 304, gels in 96-well plates.
7) Allow gels to sit for 1 hour in a cell culture incubator maintained at 5% CO2 and 37° C. Add appropriate co-culture media to wells after 1 hour. For NK-92 cells co-cultured with tumor cell lines, use NK-92 cell media.

Antibody dependent cell mediated cytotoxicity assay with mouse or human NK cells and mouse or human tumor clusters. Materials:

Materials for use: Activated NK cells; Organoids isolated from mouse mammary tumors or human patient-derived xenografts; Benchtop swinging bucket centrifuge; Cell culture incubator; Microtube incubator with two blocks set to 37° C.; 24-well glass bottom plates; 96-well glass bottom plates; DMEM/F12 media (Life Technologies #10565-018); lx TrypLE (#12604013; Thermo Fisher Scientific); Phosphate buffered saline (#10010023; Thermo Fisher Scientific); 15 mL Eppendorf conical tube; Collagen Solution titrated with sodium bicarbonate or HCl to a pH of 7.4: 375 uL of 10×DMEM (#354236, Sigma), 100 uL of sodium bicarbonate, 3 mL of Rat tail Collagen I solution (#354236, Corning); Matrigel (#354230, BD Biosciences); Co-culture media for mouse NK cells and mouse mammary tumor cells: RPMI 1640 media above, 10% (vol/vol) heat inactivated fetal bovine serum, 1% (vol/vol) 100× solution penicillin/streptomycin, 10 mM HEPES buffer, 200 U/mL recombinant mouse IL-2 (402-ML-100; R&D Systems), 5 ng/mL recombinant mouse IL-15:IL-15R complex (14-8152-62; Thermo Fisher Scientific), and 2.5 nM FGF2 (#F0291, Sigma); Co-culture media for primary human NK cells and primary human tumor cells: A 50:50 mix of: NK cell primary human media, including 25 ng/mL recombinant human IL-21 and Human organoid media disclosed herein; and NK-92 cell media; Antibody to be used for ADCC; and Antibody dilution buffer containing: Phosphate buffered saline (10010023; Thermo Fisher Scientific) and 1% (vol/vol) fetal bovine solution.

Procedure:
1) Prepare the collagen solution as described in Section 4.1 or thaw Matrigel.
2) If plating in collagen, make underlays by pipetting 104, of collagen into a well of a 24-well plate or 24, of collagen into a well of a 96-well plate.
3) Prior to co-culture, incubate tumor cells in a 1:50 concentration of antibody suspended in antibody dilution buffer for 1 hour.
4) Co-culture at a density of 30 tumor cells to 1 NK cell. Add tumor and NK cells suspended in their appropriate volume into 15 mL Eppendorf conical tube.
5) Centrifuge cell mixture at 350 g for 10 minutes.
6) Aspirate supernatant and leave cell pellet.

### Example 4: Natural Killer (NK) Cell Isolation, Activation, and Expansion, Usually Prepared One Day Before Co-Culture with Tumor Cells

Isolation of Mouse NK Cells can Come from Several Sources, Including from the Spleen and or from Tumors. Isolation of Human NK Cells can Come from Several Sources, Including Fresh or Frozen Peripheral Blood Mononuclear Cell Samples (PBMCs) from Healthy or Cancer Patient Donors. Human NK-92 Cells are Cultured and Expanded from the Cell Line NK-92.

Isolation of total immune cells from tissue requires mechanical disruption of source material to liberate cells from surrounding connective tissue. For example, dissected spleen or tumors are first mechanically disrupted before isolating immune cells from each source material.

Isolation of NK cells from tumors requires an additional separation step (e.g. centrifugation) that separates immune populations from epithelial cell populations.

Total immune cell suspensions from mouse or human sources are typically treated with a red blood cell lysis solution to remove red blood cells from the cell suspension.

NK cells are isolated from a total immune suspension by expression or absence of cell type specific molecular markers. This could be accomplished by FACS, MACS, or other methods and can rely on positive selection for NK markers or negative selection for other immune markers. A convenient method is to use a magnetic negative selection protocol that is using antibodies against specific cell surface antigens on non-NK cells and crosslinking these to magnetic beads. Then, an electromagnetic field is applied to remove non-NK cells and NK cells are left in the solution.

In specific instances such as isolation of NK cells from tumors or isolation of NK cells from human PBMCs, a positive selection is applied to assure purity of the NK cell population. For mice, key makers are CD3 and CD49b. For human, key markers are CD3 and CD56. A fluorescence-activated cell sorting machine allows for positive selection of functional cells that can be used for downstream experiments.

NK-92 cells are cultured and expanded in specific NK-92 media and this media is changed every 3-4 days.

The composition of culture, activating, and expansion NK cell media depends on the source species and tissue.

### Example 5: Preparation of Tumor Cells from Mouse or Human Sources

Isolate tumor organoids or tumor clusters on the day of co-culture. Isolation of tumor organoids from mouse or human sources involve a mechanical disruption of the source to disrupt the connective tissue. Mouse sources of tumor organoids can come from xenografts or spontaneously occurring tumors from genetically engineered mouse models. Human sources can come directly from surgical samples or from patient-derived xenografts implanted subcutaneously in mice.

Enzymatic digestion is a convenient method to separate the tissue into constituent cells and organoids and typically uses enzymes like collagenase, trypsin, and DNase followed by fast and short differential centrifugation steps allow for continued tumor digestion and stromal depletion, ultimately leading to enrichment of epithelial organoids that capture the heterogeneity of bulk tumors.

Isolated tumor organoids can be further digested down to 2-3 cell tumor clusters using an additional enzymatic step (e.g. trypsinization) and filtering step.

Tumor cells are stored in specific media that is dependent on species source until time of co-culture.

### Example 6. Co-Culture of Activated NK Cells with Tumor Cells

Co-culture of NK cells and tumor cells can be done in suspension or in a 3D environment. NK cells and tumor cells can be co-cultured in suspension. This variation of co-culture allows for easy accessibility of cell populations for separation after co-coculture and additional downstream analysis or experimentation.

NK cells and tumor cells can be co-cultured in a 3D environment of natural extracellular matrix or engineered scaffolds. It is convenient to use collagen I or Matrigel. This allows for modeling of specific tumor properties related to the metastatic process and how NK cells interact with these properties. Additionally, these also model how NK cells respond to chemoattractants produced in local microenvironments and travel through tissue towards target cells.

The use of 3D culture enables the testing of how immune cells control and interact with specific metastatic properties of tumor cells in ex vivo settings that previously was only able to be observed in vivo.

Placement of NK cells and tumor organoids in collagen gels allows for testing of how immune cells interact with invasive tumor cells, simulating how immune cells interact with tumor cells as they invade and disseminate from the primary tumor.

Placement of NK cells and tumor clusters in Matrigel allows for testing of how immune cells interact with metastatic clusters that arrive in distant organ sites, simulating how immune cells interact with tumor cells they grow from micrometastases to macrometastases.

Co-cultures are grown in 50/50 media mix of NK cell specific media and tumor cell growth media.

Co-culture gels can be digested and cell populations can be isolated and separated after a specified period of time.

The use of fluorescently labeled NK cells and tumor cells allows and separation of each population for additional functional studies and downstream analysis after a specified period of time.

Cells can be pre-treated with biologic, radiologic, and/or pharmacologic agents prior to co-culture and co-cultures can be treated with biologic, radiologic, and/or pharmacologic agents to test the effects of these agents on cell populations and the cytotoxicity of effector cells.

These 3D co-cultures have been used to show how the fundamental biology of NK cells can be altered in the presence of metastatic tumor cells.

The co-culture of NK cells and tumor cells can be used to test features of NK cells such as antibody-dependent cellular cytotoxicity. Antibodies need to be validated before use to determine that they do not have a NK cell autonomous effect on tumor cell viability.

Prior to co-culture, tumor cells are incubated and coated with cell surface antibodies present on tumor cells for a specified period of time. Then they are co-cultured with NK cells in 3D gels.

Antibody incubation with tumor cells prior to co-culture allows for the testing of antibody-dependent cellular cytotoxicity properties of innate immune cells.

### Example 7. Co-Culture of Activated NK Cells with Tumor Cells

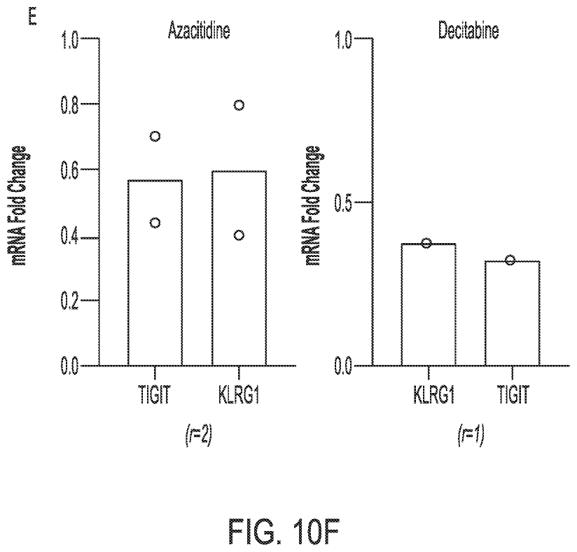

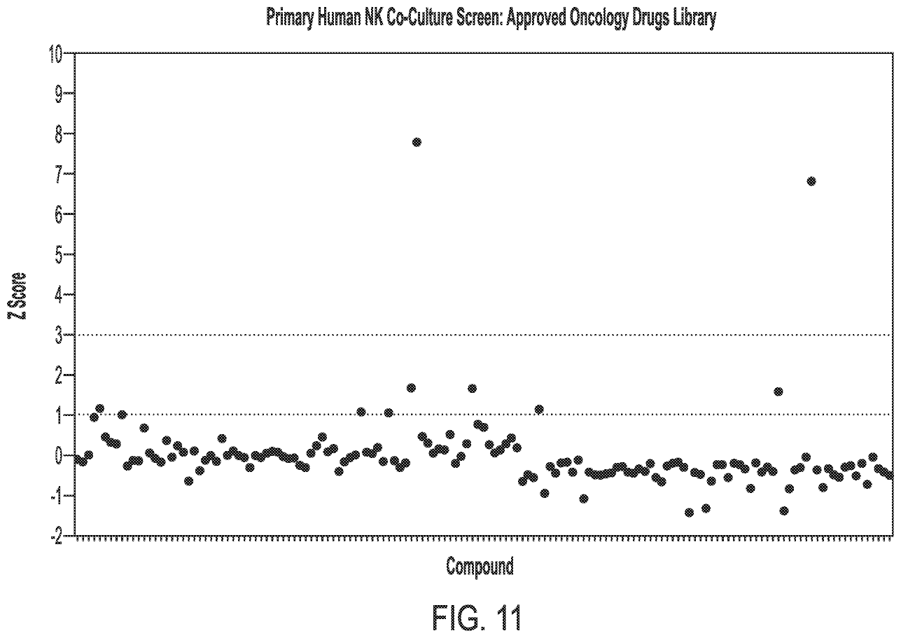

To test the effectiveness of drugs that can sensitize cancer cells to NK cell killing, cancer cells were cultured alone or in co-culture with NK cells after treatment with an anti-cancer compound library. Human cancer cell lines were cultured and a compound library of 149 FDA approved anti-cancer drugs was applied to the culture media of the cancer cells at a concentration of 1 μM. Human NK cells were isolated from a healthy human PBMC donor, expanded and activated in culture using the protocol described in Example 2. After incubation with drug for a day, media containing drug was removed. Cancer cells were then cultured in monoculture or in co-culture with NK cells. Cancer cell viability, as measured by luminescence, was compared between co-culture conditions and monoculture controls. CellTiter-Glo and a luminometer was used to measure luminescence. FIG. 11 shows the application of a chemical library to test pharmacologic perturbations that can sensitize cancer cells to NK cell killing. This example shows that the assay described herein can be used to distinguish which compounds in the library have the greatest potential utility for as NK cell-modulating therapeutics. Further, this example demonstrates that the assay can be used in a throughput application.

In this assay, specific compounds were tested that could sensitize cancer cells to NK cell killing. Using different Z-score cutoffs, targets that could sensitize cancer cells to NK cell killing were identified.

What is claimed is:

1. A composition comprising an organoid/natural killer (NK) cell co-culture, wherein the organoid/NK cell co-culture comprises one or more tumor organoids with one or more NK cells embedded in an extracellular matrix.

2. A method of identifying a drug candidate for treating cancer metastasis, the method comprising:
a) culturing an embedded organoid/natural killer (NK) cell co-culture with a drug candidate; and
b) measuring one or more metastatic properties of the embedded organoid or tumor-killing potential or tumor-promoting potential of the NK cells in the co-culture,
wherein a change in the one or more metastatic properties or an increase in the tumor killing tumor potential of the NK cells or a decrease in the tumor promoting potential in the NK cells identifies the drug candidate is capable of treating cancer metastasis.

3. A method of identifying a drug candidate that inhibits natural killer cell activity, the method comprising:
a) culturing an embedded organoid/NK cell co-culture with a drug candidate; and
b) measuring one or more metastatic properties of the embedded organoid or tumor-killing potential or tumor-promoting potential of the NK cells in the co-culture,
wherein a change in the one or more metastatic properties or an increase in the tumor killing tumor potential of the NK cells or a decrease in the tumor promoting potential in the NK cells identifies a drug candidate capable of inhibiting natural killer cell activity.

4. A method of identifying a cancer patient's responsiveness to an anticancer drug candidate, the method comprising:
a) culturing an embedded organoid/NK cell co-culture with a drug candidate; and
b) measuring one or more metastatic properties of the embedded organoid or tumor-killing potential or tumor-promoting potential of the NK cells in the co-culture,
wherein a change in the one or more metastatic properties or an increase in the tumor killing tumor potential of the NK cells or a decrease in the tumor promoting potential in the NK cells identifies an anticancer drug that the subject is responsive to.

5. A method identifying an antibody that binds to a specific tumor antigen, the method comprising:
a) culturing an embedded organoid/natural killer (NK) cell co-culture with an antibody; and
b) measuring one or more metastatic properties of the embedded organoid or tumor-killing potential or tumor-promoting potential of the NK cells in the co-culture,
wherein a change in the one or more metastatic properties or an increase in the tumor killing tumor potential of the NK cells or a decrease in the tumor promoting potential in the NK cells means that the antibody binds to the specific tumor antigen.

6. A method identifying a drug candidate for treating cancer metastasis, the method comprising:
a) culturing an embedded organoid/natural killer (NK) cell co-culture with a drug candidate; and
b) measuring one or more metastatic properties of the embedded organoid or tumor-killing potential or tumor-promoting potential of the NK cells in the co-culture,
wherein a change in the one or more metastatic properties or an increase in the tumor killing tumor potential of the NK cells or a decrease in the tumor promoting potential in the NK cells means that the antibody binds to the specific tumor antigen.

7. A method of identifying a drug candidate for treating cancer metastasis, the method comprising:
a) culturing an embedded organoid/natural killer (NK) cell co-culture with a drug candidate; and
b) measuring one or more metastatic properties of the embedded organoid or tumor-killing potential or tumor-promoting potential of the NK cells in the co-culture, wherein the one or more tumor organoids comprise one or more natural killer cells with Klrg1, TIGIT, or Lag3 present on its surface;
wherein a change in the one or more metastatic properties or an increase in the tumor killing tumor potential of the NK cells or a decrease in the tumor promoting potential in the NK cells means that the drug candidate inhibits binds to Klrg1, TIGIT, or Lag3 present on the surface of the one or more natural killer cells and wherein the change in the one or more metastatic properties or an increase in the tumor killing tumor potential of the NK cells or a decrease in the tumor promoting potential in the NK cells indicates that the subject is responsive to treatment with the drug.

8. The method of any of claims 2-7, further comprising culturing one or more tumor organoids with one or more natural killer (NK) cells to provide a organoid/NK cell co-culture and embedding the organoid/NK cell co-culture in an extracellular matrix to provide an embedded organoid/NK cell co-culture prior to step a).

9. The method of any of the preceding claims, wherein the one or more metastatic properties measured metabolic activity, cell proliferation, apoptosis, cancer invasion, or molecular or cellular correlates of metastatic ability.

10. The method of any of the preceding claims, wherein activity of the NK cells is measures cellular or molecular means.

11. The method of claim 10, wherein the molecular means is qRT-PCR.

12. The method of any of the preceding claims, wherein the tumor organoids were generated from an isolated primary tumor from a subject or patient.

13. The method of claim 12, wherein the tumor organoids were generated via mechanical disruption and/or enzymatic digestion.

14. The method of any of claims 2 to 7, wherein the drug candidate is an immune receptor inhibitor or an immune receptor activator.

15. The method of claim 2 or 5, wherein the drug candidate is an antibody.

16. The method of any of the preceding claims, wherein the one or more tumor organoids comprise one or more natural killer cells with Klrg1, TIGIT, or Lag3 present on its surface.

17. The method of any of the preceding claims, wherein the one or more organoids are generated from primary human cells.

18. The method of any of the preceding claims, wherein the one or more organoids are mammary tumor organoids.

19. The method of any of the preceding claims, wherein the change in the one or more metastatic properties or the increase in the tumor killing tumor potential of the NK cells or the decrease in the tumor promoting potential in the NK cells is determined by comparison to a reference or control organoid or NK cell.

20. The method of any of the preceding claims, wherein the drug candidates are from a library of compounds.


SEQUENCE LISTING (4 sequences)

SEQ ID NO: 1 - DNA, 23 bp, Artificial Sequence
  Synthetic construct
gctcacatctccttacatttccg

SEQ ID NO: 2 - DNA, 19 bp, Artificial Sequence
  Synthetic construct
tcctcaagccgatccagta

SEQ ID NO: 3 - DNA, 18 bp, Artificial Sequence
  Synthetic construct
ctgccttcctcgctacag

SEQ ID NO: 4 - DNA, 22 bp, Artificial Sequence
  Synthetic construct
gtaagatgacagagccaccttc